(19) United States
(12) Patent Application Publication
(10) Pub. No.: US 2024/0285907 A1
(43) Pub. Date: Aug. 29, 2024

(54) CATHETER HANDLE WITH A LOCKING MECHANISM

(71) Applicant: MEDTRONIC, INC., Minneapolis, MN (US)

(72) Inventors: Alan MCGUINN, Oranmore (IE); Niall DUFFY, Ballybrit (IE)

(21) Appl. No.: 18/569,747

(22) PCT Filed: Aug. 10, 2022
(86) PCT No.: PCT/IB2022/057472

§ 371 Date: Dec. 13, 2023

Related U.S. Application Data
(60) Provisional application No. 63/232,266, filed on Aug. 12, 2021.

(51) Int. Cl. A61M 25/01

(52) U.S. Cl. CPC A61M 25/0136

(57) ABSTRACT

A system (100) includes a handle (130), a sheath (122) attached to and distally extending from the handle (130), and a tubular component (102) slidingly disposed within the sheath (122). The handle (130) includes a locking mechanism (140) including an unlocked state in which the tubular component (102) is permitted to slide and rotate freely relative to the sheath (122), a locked state in which the tubular component (102) is not permitted to slide or rotate relative to the sheath (122), a first semi-locked state in which the tubular component (102) is permitted to slide freely relative to the sheath (122) and is not permitted to rotate freely in a circumferential direction relative to the sheath (122), and a second semi-locked state in which the tubular component (102) is not permitted to slide freely in a longitudinal direction relative to the sheath (122) and is permitted to rotate freely in a circumferential direction relative to the sheath (122).

### FIELD

Embodiments hereof relate to catheters and more particularly to a delivery system having a torqueable catheter shaft.

### BACKGROUND

A variety of catheters for delivering a therapy and/or monitoring a physiological condition have been implanted or proposed for implantation in patients. Catheters may deliver therapy to, and/or monitor conditions associated with, the heart, muscle, nerve, brain, stomach or other organs or tissue. Many catheters are tracked through the vasculature to locate a therapeutic or diagnostic portion of the catheter at a target site. Such catheters must have flexibility to navigate the twists and turns of the vasculature, sufficient stiffness in the proximal portion thereof to be pushed through the vasculature alone or over a guidewire or through a lumen, and the capability of orienting a distal portion thereof in alignment with an anatomical feature at the target site so that a diagnostic or therapeutic procedure can be completed. In general terms, the catheter body must also resist kinking and be capable of being advanced through access pathways that twist and turn, sometimes abruptly at acute angles.

For certain procedures, it may be necessary for the clinician to accurately steer or deflect the catheter so that a distal opening thereof may be aligned with an ostium of a branch or side vessel. The distal portions of catheters frequently need to be selectively curved or bent and straightened again while being advanced within the patient to steer the catheter distal end into a desired body lumen or chamber. For example, it may be necessary to direct the catheter distal end through tortuous anatomies and/or into a branch at a vessel bifurcation. In addition, some procedures require high accuracy in guidewire orientation. For example, often patient's arteries are irregularly shaped, highly tortuous and very narrow. The tortuous configuration of the arteries may present difficulties to a clinician in advancement of a catheter to a treatment site.

In addition to bending or deflecting the distal portion of the catheter during navigation, the clinician may also need to rotate or torque the catheter when advancing the catheter to a treatment site in order to achieve proper or desired alignment of the catheter. However, manually torqueing the delivery system may require significant force to combat recoiling forces.

Thus, a need in the art still generally exists for improved apparatuses and methods for navigating a catheter through or within a patient's anatomy.

### BRIEF SUMMARY

According to a first embodiment hereof, the present disclosure provides a system which includes a handle, a sheath attached to and distally extending from the handle, and a tubular component slidingly disposed within the sheath. The handle includes a locking mechanism including an unlocked state in which the tubular component is permitted to slide freely in a longitudinal direction relative to the sheath and is permitted to rotate freely in a circumferential direction relative to the sheath, a locked state in which the tubular component is not permitted to slide freely in the longitudinal direction relative to the sheath and is not permitted to rotate freely in the circumferential direction relative to the sheath, a first semi-locked state in which the tubular component is permitted to slide freely in a longitudinal direction relative to the sheath and is not permitted to rotate freely in a circumferential direction relative to the sheath, and a second semi-locked state in which the tubular component is not permitted to slide freely in a longitudinal direction relative to the sheath and is permitted to rotate freely in a circumferential direction relative to the sheath.

In an aspect of the first embodiment, and in combination with any other aspects herein, the disclosure provides that the tubular component forms an outer tubular component of a balloon catheter.

In an aspect of the first embodiment, and in combination with any other aspects herein, the disclosure provides that the locking mechanism includes a collet, a locking collar, and a locking bearing, each of the locking collar and the locking bearing being slidingly disposed over the collet.

In an aspect of the first embodiment, and in combination with any other aspects herein, the disclosure provides a locking interface on an outer surface of the tubular component. The locking interface has an outer surface including a plurality of grooves and a plurality of ridges, each groove formed between a pair of adjacent ridges. The collet is disposed over the locking interface and the collet has an inner surface with a sinusoidal configuration including a plurality of peaks and a plurality of valleys, each valley formed between a pair of adjacent peaks. Each groove of the locking interface is configured to receive a pair of adjacent peaks of the collet.

In an aspect of the first embodiment, and in combination with any other aspects herein, the disclosure provides that each of the locking collar and the locking bearing is configured to be axially translated by the user between at least a non-engaged position on the collet and an engaged position on the collet.

In an aspect of the first embodiment, and in combination with any other aspects herein, the disclosure provides that each of the locking collar and the locking bearing is disposed in the non-engaged position on the collet when the locking mechanism is in the unlocked state.

In an aspect of the first embodiment, and in combination with any other aspects herein, the disclosure provides that each of the locking collar and the locking bearing is disposed in the engaged position on the collet when the locking mechanism is in the locked state.

In an aspect of the first embodiment, and in combination with any other aspects herein, the disclosure provides that the locking collar is disposed in the engaged position and the locking bearing is disposed in the non-engaged position on the collet when the locking mechanism is in the first semi-locked state.

In an aspect of the first embodiment, and in combination with any other aspects herein, the disclosure provides that the locking collar is disposed in the non-engaged position on the collet and the locking bearing is disposed in the engaged position on the collet when the locking mechanism is in the second semi-locked state.

In an aspect of the first embodiment, and in combination with any other aspects herein, the disclosure provides that the handle further includes an indexing component configured to output a tactile click at one or more defined degrees of rotation of the treatment catheter.

According to a second embodiment hereof, the present disclosure provides a system which includes a treatment catheter and a guide catheter including a sheath configured to receive the treatment catheter and a handle coupled to the sheath. The handle includes a locking mechanism having an unlocked state in which the treatment catheter is permitted to slide freely in a longitudinal direction relative to the sheath and is permitted to rotate freely in a circumferential direction relative to the sheath, a locked state in which the treatment catheter is not permitted to slide freely in the longitudinal direction relative to the sheath and is not permitted to rotate freely in the circumferential direction relative to the sheath, a first semi-locked state in which the treatment catheter is permitted to slide freely in a longitudinal direction relative to the sheath and is not permitted to rotate freely in a circumferential direction relative to the sheath, and a second semi-locked state in which the treatment catheter is not permitted to slide freely in a longitudinal direction relative to the sheath and is permitted to rotate freely in a circumferential direction relative to the sheath.

In an aspect of the second embodiment, and in combination with any other aspects herein, the disclosure provides that an outer surface of the treatment catheter includes a locking interface disposed thereon. The locking interface has an outer surface including a plurality of grooves and a plurality of ridges, each groove formed between a pair of adjacent ridges. The locking mechanism is disposed over the locking interface and has an inner surface with a sinusoidal configuration including a plurality of peaks and a plurality of valleys, each valley formed between a pair of adjacent peaks. Each groove of the locking interface is configured to receive a pair of adjacent peaks of the locking mechanism.

In an aspect of the second embodiment, and in combination with any other aspects herein, the disclosure provides that the locking mechanism includes a collet, a locking collar, and a locking bearing, each of the locking collar and the locking bearing being slidingly disposed over the collet.

In an aspect of the second embodiment, and in combination with any other aspects herein, the disclosure provides that the collet includes the inner surface including the plurality of peaks and the plurality of valleys.

In an aspect of the second embodiment, and in combination with any other aspects herein, the disclosure provides that each of the locking collar and the locking bearing is configured to be axially translated by the user between at least a non-engaged position on the collet and an engaged position on the collet.

In an aspect of the second embodiment, and in combination with any other aspects herein, the disclosure provides that the each of the locking collar and the locking bearing is disposed in the non-engaged position on the collet when the locking mechanism is in the unlocked state.

In an aspect of the second embodiment, and in combination with any other aspects herein, the disclosure provides that each of the locking collar and the locking bearing is disposed in the engaged position on the collet when the locking mechanism is in the locked state.

In an aspect of the second embodiment, and in combination with any other aspects herein, the disclosure provides that the locking collar is disposed in the engaged position and the locking bearing is disposed in the non-engaged position on the collet when the locking mechanism is in the first semi-locked state.

In an aspect of the second embodiment, and in combination with any other aspects herein, the disclosure provides that the locking collar is disposed in the non-engaged position on the collet and the locking bearing is disposed in the engaged position on the collet when the locking mechanism is in the second semi-locked state.

In an aspect of the second embodiment, and in combination with any other aspects herein, the disclosure provides that the handle further includes an indexing component configured to output a tactile click at one or more defined degrees of rotation of the treatment catheter.

According to a third embodiment hereof, the present disclosure provides a system including a catheter and a locking mechanism. An outer surface of the catheter includes a locking interface having an outer surface including a plurality of grooves and a plurality of ridges, each groove formed between a pair of adjacent ridges. The locking mechanism is disposed over the locking interface. The locking mechanism includes a collet, a locking collar, and a locking bearing. Each of the locking collar and the locking bearing is slidingly disposed over the collet. The collet has an inner surface with a sinusoidal configuration including a plurality of peaks and a plurality of valleys, each valley formed between a pair of adjacent peaks. Each groove of the locking interface is configured to receive a pair of adjacent peaks of the collet. At least one of the locking collar and the locking bearing is configured to be axially translated by the user between at least a non-engaged position on the collet and an engaged position on the collet.

In an aspect of the third embodiment, and in combination with any other aspects herein, the disclosure provides that the catheter is a balloon catheter.

In an aspect of the third embodiment, and in combination with any other aspects herein, the disclosure provides that each of the locking collar and the locking bearing is configured to be axially translated by the user between at least a non-engaged position on the collet and an engaged position on the collet.

In an aspect of the third embodiment, and in combination with any other aspects herein, the disclosure provides that only the locking collar is configured to be axially translated by the user between at least a non-engaged position on the collet and an engaged position on the collet.

In an aspect of the third embodiment, and in combination with any other aspects herein, the disclosure provides that the locking bearing is disposed on the collet in an engaged position.

In an aspect of the third embodiment, and in combination with any other aspects herein, the disclosure provides that only the locking bearing is configured to be axially translated by the user between at least a non-engaged position on the collet and an engaged position on the collet.

In an aspect of the third embodiment, and in combination with any other aspects herein, the disclosure provides that the locking collar is disposed on the collet in an engaged position.

In an aspect of the third embodiment, and in combination with any other aspects herein, the disclosure provides that the locking collar is disposed on the collet in a non-engaged position.

In an aspect of the third embodiment, and in combination with any other aspects herein, the disclosure provides that the locking mechanism is disposed within a handle of a guide catheter and a sheath of the guide catheter is configured to slidingly receive the catheter.

In an aspect of the third embodiment, and in combination with any other aspects herein, the disclosure provides that the handle further includes an indexing component configured to output a tactile click at one or more defined degrees of rotation of the catheter.

According to a fourth embodiment hereof, the present disclosure provides a catheter configured for passing through vasculature to a target site. The catheter includes a proximal end portion and a distal end portion. The distal end portion includes a medical component and the proximal end portion has an outer surface. The outer surface has a plurality of grooves and at least one raised portion.

In an aspect of the fourth embodiment, and in combination with any other aspects herein, the disclosure provides that the outer surface forms an interface for coupling to a control apparatus.

In an aspect of the fourth embodiment, and in combination with any other aspects herein, the disclosure provides that the outer surface has a plurality of longitudinally extending grooves and the raised portion includes a longitudinally extending ridge extending between adjacent grooves.

In an aspect of the fourth embodiment, and in combination with any other aspects herein, the disclosure provides that the outer surface has a plurality of longitudinally extending grooves and a plurality of said raised portions. Each raised portion includes a longitudinally extending ridge extending between adjacent grooves.

In an aspect of the fourth embodiment, and in combination with any other aspects herein, the disclosure provides that the outer surface is integrally formed with the catheter.

In an aspect of the fourth embodiment, and in combination with any other aspects herein, the disclosure provides that the catheter includes a first tubular member and a second member that forms the outer surface. The first and second members are fixedly secured to one another.

In an aspect of the fourth embodiment, and in combination with any other aspects herein, the disclosure provides that the catheter is a treatment catheter and the medical component is a balloon.

In an aspect of the fourth embodiment, and in combination with any other aspects herein, the disclosure provides that the raised portion is a longitudinally extending ridge.

In an aspect of the fourth embodiment, and in combination with any other aspects herein, the disclosure provides that the outer surface includes a plurality of grooves and the raised portion extends longitudinally between adjacent grooves.

In an aspect of the fourth embodiment, and in combination with any other aspects herein, the disclosure provides that the catheter is a diagnostic catheter and the medical component is a sensor.

According to a fifth embodiment hereof, the present disclosure provides a catheter configured for passing through vasculature to a target site. The catheter includes a proximal end portion and a distal end portion. The distal end portion includes a medical component and the proximal end portion has an outer surface. The outer surface has a plurality of ridges and a groove extending between adjacent ridges.

In an aspect of the fifth embodiment, and in combination with any other aspects herein, the disclosure provides that the outer surface forms an interface for coupling to a control apparatus.

In an aspect of the fifth embodiment, and in combination with any other aspects herein, the disclosure provides that the outer surface has a plurality of longitudinally extending grooves and a longitudinally extending ridge extending between adjacent grooves.

In an aspect of the fifth embodiment, and in combination with any other aspects herein, the disclosure provides that the outer surface has a plurality of longitudinally extending grooves and a plurality of longitudinally extending ridges, each longitudinally extending ridge extending between adjacent grooves.

In an aspect of the fifth embodiment, and in combination with any other aspects herein, the disclosure provides that the outer surface is integrally formed with the catheter.

In an aspect of the fifth embodiment, and in combination with any other aspects herein, the disclosure provides that the catheter includes a first tubular member and a second member that forms the outer surface. The first and second members being fixedly secured to one another.

In an aspect of the fifth embodiment, and in combination with any other aspects herein, the disclosure provides that the catheter is a treatment catheter and the medical component is a balloon.

In an aspect of the fifth embodiment, and in combination with any other aspects herein, the disclosure provides that the catheter is a diagnostic catheter and the medical component is a sensor.

### BRIEF DESCRIPTION OF DRAWINGS

The foregoing and other features and advantages of the invention will be apparent from the following description of embodiments hereof as illustrated in the accompanying drawings. The accompanying drawings, which are incorporated herein and form a part of the specification, further serve to explain the principles of the invention and to enable a person skilled in the pertinent art to make and use the invention. The drawings are not to scale.

FIG. 1 is a side view of a system according to an embodiment hereof, wherein a balloon-expandable prosthesis of the system is shown in a delivery or unexpanded configuration.

FIG. 1A is a cross-sectional view taken along line A-A of FIG. 1.

FIG. 2 is a side view of the system of FIG. 1, wherein the balloon-expandable prosthesis of the system is shown in a deployed or expanded configuration.

FIG. 3 is a perspective view of a locking mechanism of the system of FIG. 1, wherein the locking mechanism is removed from a handle of the system for sake of illustration.

FIG. 3A is a sectional view taken along line A-A of FIG. 3.

FIG. 4 is an end view of the locking mechanism of FIG. 3.

FIG. 5 is another end view of the locking mechanism of FIG. 3.

FIG. 6 is a perspective view of a collet of the locking mechanism of FIG. 3.

FIG. 7 is an end view of a first end of the collet of the locking mechanism of FIG. 3.

FIG. 7A is an end view of the first end of the collet and a locking interface of the locking mechanism of FIG. 3.

FIG. 8 is an end view of a second end of the collet of the locking mechanism of FIG. 3.

FIG. 8A is an end view of the second end of the collet and a locking interface of the locking mechanism of FIG. 3.

FIG. 9 is an end view of a locking collar of the locking mechanism of FIG. 3.

FIG. 9A is a side view of the collet and the locking collar, wherein the non-engaged position of the locking collar is shown.

FIG. 9B is a side view of the collet and the locking collar, wherein the engaged position of the locking collar is shown.

FIG. 10 is an end view of a locking bearing of the locking mechanism of FIG. 3.

FIG. 10A is a side view of the collet and the locking bearing, wherein the non-engaged position of the locking bearing is shown.

FIG. 10B is a side view of the collet and the locking bearing, wherein the engaged position of the locking bearing is shown.

FIG. 11 is an enlarged perspective view of a portion of the locking mechanism of FIG. 3.

FIG. 12 is an enlarged perspective view of a portion of the locking mechanism of FIG. 3.

FIG. 13 is a side view of the locking mechanism in an unlocked state, wherein axial translation and rotation of a treatment catheter of the system are both permitted.

FIG. 14 is a side view of the locking mechanism in a locked stated, wherein neither axial translation nor rotation of the treatment catheter of the system are permitted.

FIG. 15 is a side view of the locking mechanism in a first semi-locked stated, wherein axial translation of the treatment catheter is permitted and rotation of the treatment catheter is not permitted.

FIG. 16 is a side view of the locking mechanism in a second semi-locked stated, wherein axial translation of the treatment catheter is not permitted and rotation of the treatment catheter is permitted.

FIG. 17 is a perspective view of a locking mechanism of the system of FIG. 1, wherein the system further includes an indexing component according to an embodiment hereof and wherein the locking mechanism is removed from a handle of the system for sake of illustration.

FIG. 18 is a perspective view of a locking mechanism of the system of FIG. 1, wherein the system further includes an indexing component according to another embodiment hereof and wherein the locking mechanism is removed from a handle of the system for sake of illustration.

FIG. 19 is a perspective view of a locking bearing according to another embodiment hereof.

FIG. 20 is a perspective view of a locking interface according to another embodiment hereof.

FIG. 21 is a perspective end view of a locking mechanism including the locking interface of FIG. 20.

FIG. 22 is another perspective end view of the locking mechanism of FIG. 21.

FIG. 23 is a perspective end view of a collet according to another embodiment hereof.

FIG. 24 is another perspective end view of the collet of FIG. 23.

FIG. 25 is a perspective end view of a locking mechanism including the collet of FIG. 23.

FIG. 26 is another perspective end view of the locking mechanism of FIG. 25.

### DETAILED DESCRIPTION

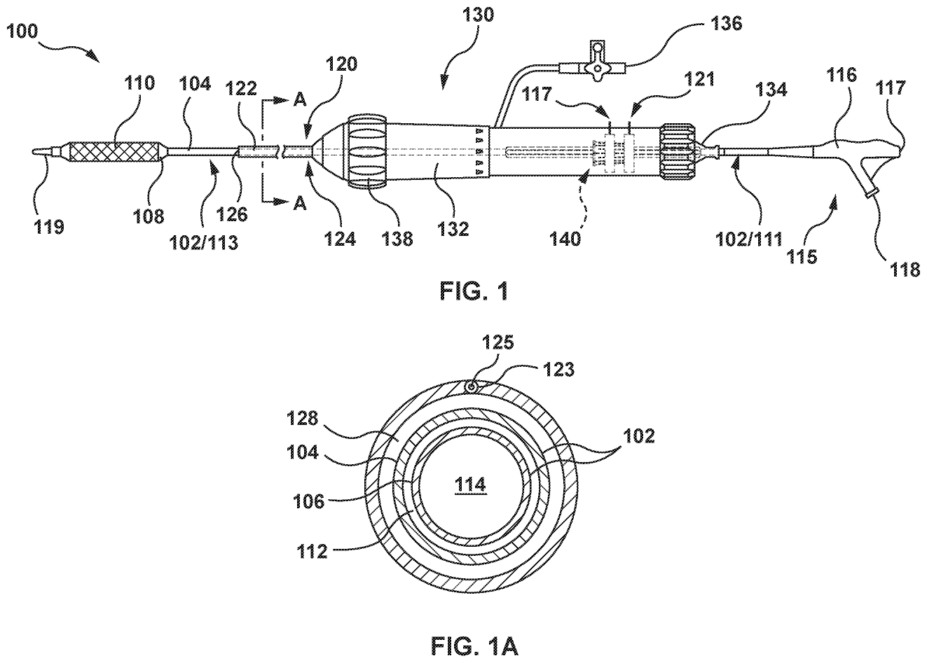

Specific embodiments of the present invention are now described with reference to the figures, wherein like reference numbers indicate identical or functionally similar elements. The terms “distal” and “proximal” are used in the following description with respect to a position or direction relative to the treating clinician. “Distal” or “distally” are a position distant from or in a direction away from the clinician. “Proximal” and “proximally” are a position near or in a direction toward the clinician. In addition, “slidably” or “slidable” denotes back and forth movement in a longitudinal direction about a longitudinal axis LA of the system (shown in FIG. 1) while “rotatably” or “rotatable” denotes movement or rotation about the longitudinal axis LA.

The following detailed description is merely exemplary in nature and is not intended to limit the invention or the application and uses of the invention. Although the description of the invention is in the context of delivery of a balloon-expandable prosthesis, the invention may also be used where it is deemed useful in endoscopic procedures, procedures in the coronary vessels, or procedures in the peripheral vessels. Furthermore, there is no intention to be bound by any expressed or implied theory presented in the preceding technical field, background, brief summary or the following detailed description.

Embodiments hereof relate to a handle with a locking mechanism configured to selectively release a tubular component disposed within the handle in order to allow the user to rotate and/or axially translate the tubular component relative to the handle. In an embodiment, the handle is part of a guide catheter that also includes a sheath extending distally from the handle, and the tubular component disposed within the handle is part of a treatment catheter such as but not limited to a balloon catheter or a valve delivery catheter. The locking mechanism of the handle permits a user to selectively rotate and/or axially translate the treatment catheter relative to the sheath and the handle of the guide catheter. The locking mechanism includes an unlocked state or configuration in which the tubular component is permitted to slide freely in a longitudinal direction relative to the handle and is permitted to rotate freely in a circumferential direction relative to the handle, a locked state or configuration in which the tubular component is not permitted to slide freely in the longitudinal direction relative to the handle and is not permitted to rotate freely in the circumferential direction relative to the handle, a first semi-locked state or configuration in which the tubular component is permitted to slide freely in a longitudinal direction relative to the handle and is not permitted to rotate freely in a circumferential direction relative to the handle, and a second semi-locked state or configuration in which the tubular component is not permitted to slide freely in a longitudinal direction relative to the handle and is permitted to rotate freely in a circumferential direction relative to the handle. Stated another way, the locking mechanism of the handle may be considered a toggle switch that enables switching between four different states or configurations: (1) an unlocked state in which the treatment catheter is permitted move freely in both longitudinal and rotational directions relative to the guide catheter, (2) a locked state in which the treatment catheter is locked such that rotational and longitudinal movement relative to the guide catheter is prevented, (3) a first semi-locked state in which longitudinal movement of the treatment catheter relative to the guide catheter is permitted while preventing longitudinal movement thereof, and (4) a second semi-locked state in which rotational movement of the treatment catheter relative to the guide catheter is permitted while preventing longitudinal movement thereof. Controlled longitudinal and rotational movement of the treatment catheter relative to the guide catheter is beneficial for precise positioning of the treatment catheter in situ.

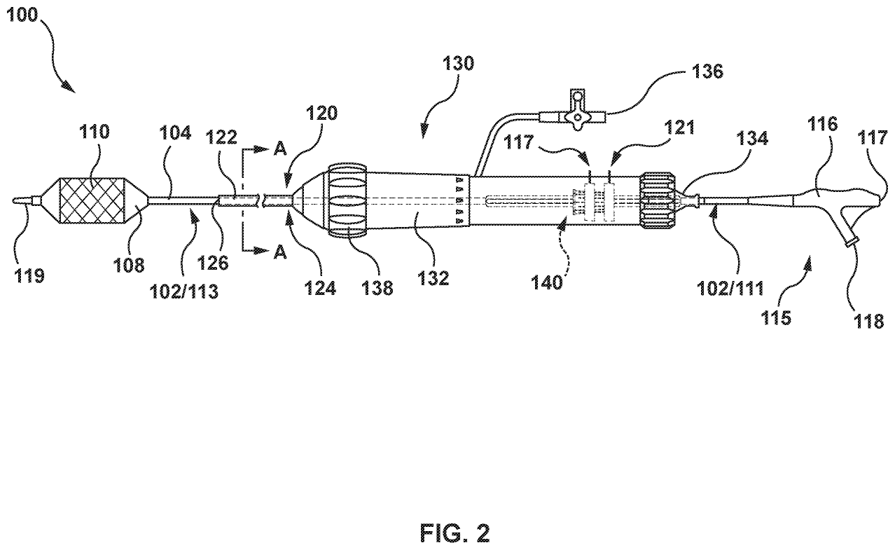

FIGS. 1, 1A, and 2 illustrates a system 100 according to an embodiment hereof. The system 100 includes a treatment catheter 102 and a guide catheter 120. The guide catheter 120 includes a sheath 122 configured to receive the treatment catheter 102 and a handle 130 coupled to the sheath 122. As will be explained in more detail herein, the handle 130 includes a locking mechanism 140 having an unlocked state or configuration in which the treatment catheter 102 is permitted to slide freely in a longitudinal direction relative to the sheath 122 and is permitted to rotate freely in a circumferential direction relative to the sheath 122, a locked state or configuration in which the treatment catheter 102 is not permitted to slide freely in the longitudinal direction relative to the sheath 122 and is not permitted to rotate freely in the circumferential direction relative to the sheath 122, a first semi-locked state or configuration in which the treatment catheter 102 is permitted to slide freely in a longitudinal direction relative to the sheath 122 and is not permitted to rotate freely in a circumferential direction relative to the sheath, and a second semi-locked state or configuration in which the treatment catheter 102 is not permitted to slide freely in a longitudinal direction relative to the sheath 122 and is permitted to rotate freely in a circumferential direction relative to the sheath 122.

The sheath 122 of the guide catheter 120 is an elongated tubular component sized for insertion into a lumen, such as a blood vessel, within the human body. The sheath 122 of the guide catheter 120 has a proximal end 124 that extends outside of a patient, a distal end 126, and defines a lumen 128 therethrough which other elements such as the treatment catheter 102 may be inserted. The handle 130 is coupled to the proximal end 124 of the sheath 122 and may be manipulated by an operator. The sheath 122 of the guide catheter 120 may be formed of a polymeric material, non-exhaustive examples of which include polyethylene, PEBA, polyamide and/or combinations thereof, either blended or co-extruded. Optionally, the sheath 122 of the guide catheter 120 or some portion thereof may be formed as a composite having a reinforcement material incorporated within a polymeric body in order to enhance strength and/or flexibility. Suitable reinforcement layers include braiding, wire mesh layers, embedded axial wires, embedded helical or circumferential wires, and the like.

The proximal end 124 of the sheath 122 extends into the handle 130 and is coupled thereto. The handle 130 includes a housing 132, a strain relief component 134 at a proximal end thereof, a flush port 136, an actuator 138 for controlling a steering mechanism of the guide catheter 102, and the locking mechanism 140. In an embodiment, the actuator 138 controls tensioning of one or more pull wires 125 (shown in the cross-sectional view of FIG. 1A) attached to the sheath 122. The pull wire 125 is operable to bend the sheath 122 for steering the guide catheter 120 in situ and may be selectively tensioned via the user. The pull wire 125 may extend through a pull wire lumen 123 that extends adjacent or parallel to the lumen 128, and terminates proximal to the distal end 126 of the sheath 122. More particularly, the pull wire 125 is slidably disposed within the pull wire lumen 123 such that it may be selectively tensioned by the user to bend the distal portion of the guide catheter 120. As used herein, “slidably” denotes back and forth movement in a longitudinal direction along or generally parallel to a central longitudinal axis LA of the assembly 100. While the pull wire 125 is primarily housed or disposed within the pull wire lumen 123 of the sheath 122, the proximal end thereof (not shown) proximally extends beyond the proximal end 124 of the sheath 122 and is accessible via the handle 130 to be pulled or pushed which results in controlled bending movement of the distal portion of the sheath 122.

A proximal end of the pull wire 125 is coupled to the actuator 138 of the handle 130 and a distal end of the pull wire 125 is attached to the sidewall of the sheath 122, adjacent to the distal end 126 of the sheath 122. The pull wire 125 is thus accessible to a user via the actuator 138 of the handle 130 and the curvature of the distal portion of the guide catheter 120 can be changed based on the user manipulating the pull wire 125 via the actuator 138 of the handle 130. In the embodiment depicted in FIG. 1, the actuator 138 is a knob that is rotatable relative to the housing 132 of the handle 130. When the knob is rotated in a first direction (i.e., one of clockwise or counter-clockwise), the pull wire 125 is retracted and placed under tension to bend or deflect the distal portion of the sheath 122. Stated another way, when the pull wire 125 is retracted via actuation of the actuator 138, the pull wire 125 is placed under tension and bends the distal portion of the sheath 122. When the knob is rotated in a second direction opposite from the first direction (i.e., the other of clockwise or counter-clockwise), tension on the pull wire 125 is released and the distal portion of the sheath 122 resumes its straightened configuration. Accordingly, the distal portion of the guide catheter 120 may be bent in a first direction via activation of the pull wire 125. If it is desired to bend or deflect the distal portion of the guide catheter 120 in an opposing direction (i.e., a second direction opposite from the first direction), the guide catheter 120 may be torqued or rotated approximately 180 degrees and then the pull wire 125 may be actuated to bend the distal portion of the guide catheter 120. The dimension of the radius of curvature depends upon the intended application of the guide catheter 120, the target anatomy for use of the guide catheter 120, and/or the size or profile of the guide catheter 120.

In the embodiment depicted in FIGS. 1, 1A, and 2, the treatment catheter 102 is a balloon catheter having an inflatable balloon 108 for radially expanding a balloon-expandable prosthesis 110 mounted on the balloon 108. The balloon-expandable prosthesis 110 is shown in its delivery or unexpanded configuration in FIG. 1 and is shown in an expanded or deployed configuration in FIG. 2. As will be understood by those of ordinary skill in the art, the balloon-expandable prosthesis 110 is radially expanded or deployed in situ by the balloon 108 and released from the treatment catheter 102 at a desired location in a patient's body lumen. An exemplary balloon-expandable prosthesis, and the expanded or deployed configuration thereof, is described in more detail in U.S. patent application Ser. No. 16/778,688, filed Jan. 31, 2020, herein incorporated by reference in its entirety. However, the configuration of the balloon-expandable prosthesis 110 is merely exemplary, and it would be apparent to one of ordinary skill in the art that the treatment catheter 102 may be utilized for delivering and deploying various types or configurations of prostheses. Further, although depicted as a balloon catheter for delivering the balloon-expandable prosthesis 110, the treatment catheter 102 is not required to be configured for delivering a prosthesis but rather the treatment catheter 102 may be utilized in other procedures or for other purposes including diagnostic purposes. In addition, although embodiments hereof are shown with the balloon-expandable prosthesis 110 mounted over the balloon 108 in a delivery configuration, the balloon-expandable prosthesis 110 may be mounted proximal to the balloon 108 during delivery and the balloon 108 may be proximally retracted in situ to position the balloon 108 under the balloon-expandable prosthesis 110 for deployment thereof.

The treatment catheter 102 includes a proximal portion 111 that extends out of the patient during clinical use and has a handle 115. As would be understood by one of ordinary skill in the art of balloon catheter design, the handle 115 includes a bifurcated luer 116 or other type of fitting that may be connected to a source of inflation fluid and may be of another construction or configuration without departing from the scope of the present invention. A distal portion 113 of the treatment catheter 102 is positionable at a target treatment location and includes the balloon 108, which is shown in an unexpanded configuration in FIG. 1 and is shown in an expanded or inflated configuration in FIG. 2. Stated another way, the balloon 108 is expandable from a first diameter shown in FIG. 1 to a second diameter shown in FIG. 2, the second diameter being greater than the first diameter. The balloon 108 may be made of a biocompatible material such as a thermoplastic polyurethane (TPU) resin, styrene-ethylene-butadiene-styrene (SEBS), PEBAX®, or other suitable polymeric material used for dilatation balloon manufacturing.

With reference to the FIG. 1A which is a cross-sectional view taken along line A-A of FIG. 1, the treatment catheter 102 may have an over-the-wire coaxial catheter configuration with an outer tubular component or outer shaft 104 and an inner tubular component or inner shaft 106. A proximal end of the outer shaft 104 is coupled to the handle 115 and a distal end of the outer shaft 104 is coupled to a proximal end of the balloon 108. The inner shaft 106 is disposed through the outer shaft 104. The inner shaft 106 defines a guidewire lumen 114 extending substantially the entire length of the treatment catheter 102 for accommodating a guidewire (not shown) such that the treatment catheter 102 may be slidingly disposed and tracked over the guidewire. The inner shaft 106 has a proximal end (obscured from view in FIGS. 1 and 2) coupled to the handle 115 and a distal end terminating distally of the balloon 108 and defining a distal guidewire port. Stated another way, the guidewire lumen 114 is open at the distal end of the inner shaft 106. A distal tip 119 is attached to the distal end of the inner shaft 106, and the distal tip 119 forms the distal end of the treatment catheter 102. The inner shaft 106 extends coaxially within the outer shaft 104 such that an annular inflation lumen 112 is defined between an inner surface of the outer shaft 104 and an outer surface of the inner shaft 106. The annular inflation lumen 112 is in fluid communication with an interior of the balloon 108, and extends through the outer shaft 104 and into the inner volume of the balloon 108 to allow inflation fluid received through an inflation port 118 of the bifurcated luer 116 of the handle 115 to be delivered to the balloon 108. A distal end of the balloon 108 is coupled to the inner shaft 106 at a position proximal to the distal end of the inner shaft.

Other types of catheter construction are also amendable to the invention, such as, without limitation thereto, a catheter shaft formed by multi-lumen profile extrusion. For example, rather than including coaxial inner and outer catheter shafts, the balloon catheter may include a single catheter shaft that defines an inflation lumen and a guidewire lumen, each extending substantially the entire length of the catheter and parallel to each other. Stated another way, the inflation and guidewire lumens may be defined or preformed in a sidewall of a single catheter shaft. In yet another embodiment, the inflation lumen may alternatively be formed via an elongated inflation tube disposed within and attached to the outer shaft 104, as will be understood by those of ordinary skill in the art of balloon catheter construction. In addition, the treatment catheter 102 may have a rapid-exchange configuration with the guidewire lumen extending only along a distal portion of the catheter, as understood by those of ordinary skill in the art.

When positioning the treatment catheter 102 in situ, it may be necessary to torque or rotate the treatment catheter 102 in order to properly position the balloon-expandable prosthesis 110 within a native annulus prior to deployment of the balloon-expandable prosthesis 110. Steering of the system 100 is accomplished via the flexing or bending of the sheath 122 and manipulation of the pull wire 125 as described above, and permits the user to navigate the system 100 through curved anatomy such as the aortic arch. Rotation or torqueing of the treatment catheter 102 assists the user in properly aligning the balloon-expandable prosthesis 110 within the target site, i.e., the native annulus. For example, the balloon-expandable prosthesis 110 needs to be properly aligned, axially and annularly/circumferentially, so that the balloon-expandable prosthesis 110 properly engages the native leaflets/tissue of the target site, e.g., the aortic annulus, without causing conduction blockages by implanting too deep or causing an embolization of the balloon-expandable prosthesis 110 because it was implanted too high. Torqueing the treatment catheter 102 is accomplished via rotation thereof, and permits the user to circumferentially align the balloon-expandable prosthesis 110 within the target site, e.g., the native annulus AN, in situ. When being positioned in situ, it is very important to avoid blocking the ostia of the right coronary artery and/or the left main coronary artery. Proper circumferential or rotational orientation within the target site reduces the risk of blocking coronary access. In addition, it may be desired to rotationally align commissures of the balloon-expandable prosthesis 110 with the native valve commissures. Commissure to commissure alignment (prosthesis commissure to native commissure) may improve hemodynamics and leaflet durability of the balloon-expandable prosthesis 110. To circumferentially align the balloon-expandable prosthesis 110, the balloon-expandable prosthesis 110 can rotated in situ to be positioned in a desired circumferential or rotational alignment.

The locking mechanism 140 of the handle 130 permits precise positioning of the treatment catheter 102 relative to the guide catheter 102. More particularly, the locking mechanism 140 permits a user to selectively rotate and/or axially translate the treatment catheter 102 in order to position the balloon-expandable prosthesis 100 as desired. The locking mechanism 140 includes an unlocked state or configuration in which the treatment catheter 102 is permitted to slide freely in a longitudinal direction relative to the guide catheter 102 and is permitted to rotate freely in a circumferential direction relative to the guide catheter 102, and also includes a locked state or configuration in which the treatment catheter 102 is not permitted to slide freely in the longitudinal direction relative to the guide catheter 120 and is not permitted to rotate freely in the circumferential direction relative to the guide catheter 120. In addition to the unlocked and locked configuration, the locking mechanism 140 further includes a first semi-locked state or configuration and a second semi-locked state or configuration. In the first semi-locked state, the treatment catheter 102 is permitted to slide freely in a longitudinal direction relative to the guide catheter 120 and is not permitted to rotate freely in a circumferential direction relative to the guide catheter 120, thereby restricting the treatment catheter 102 to only longitudinal movement in the proximal or distal direction while preventing inadvertent rotation of the treatment catheter 102. In the second semi-locked state or configuration, the treatment catheter 102 is not permitted to slide freely in a longitudinal direction relative to the guide catheter 120 and is permitted to rotate freely in a circumferential direction relative to the guide catheter 120, thereby permitting torqueing or rotation of the treatment catheter 102 while preventing inadvertent translation of the treatment catheter 102 in the proximal or distal direction. During positioning of the balloon-expandable prosthesis 110, a user may desire to only adjust the longitudinal position of the treatment catheter 102 or may desire to only adjust the circumferential position of the treatment catheter 102. The locking mechanism 140 thus allows a user to control which type of movement, i.e., longitudinal and/or circumferential, of the treatment catheter 102 is permitted and prevents any undesirable movement of the treatment catheter 102.

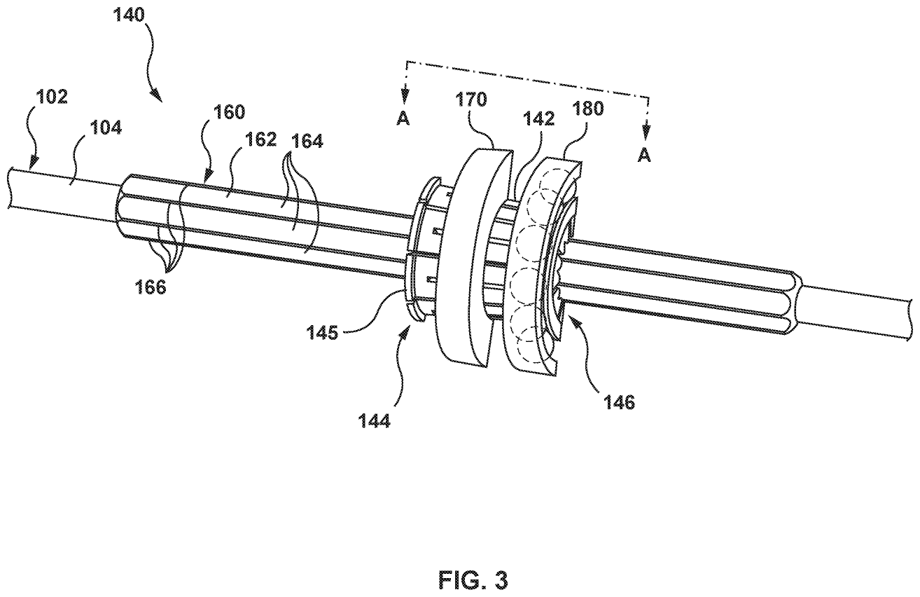

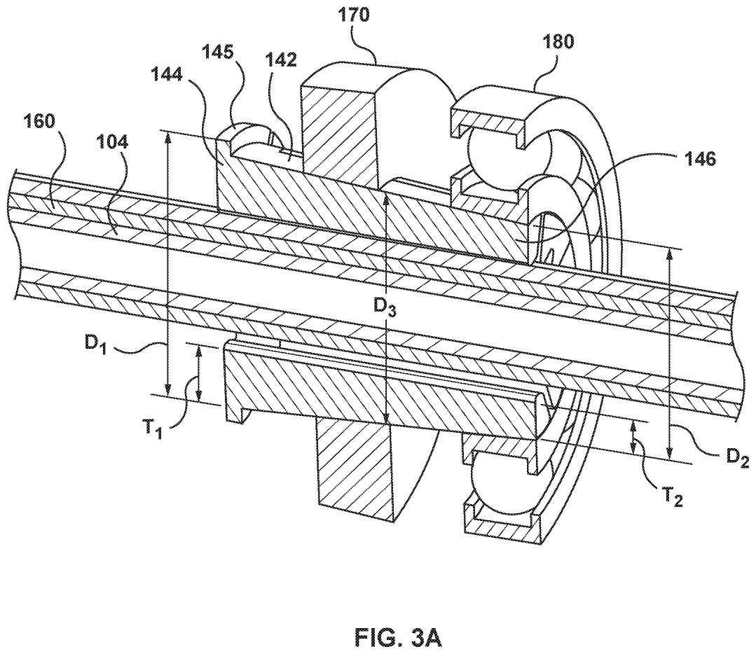

The locking mechanism 140 is described in more detail herein with reference to FIGS. 3-16. FIG. 3 is a perspective view of the locking mechanism 140 removed from the handle 130 of the system 100 for sake of illustration, and FIG. 3A is a sectional line taken along line A-A of FIG. 3. The locking mechanism 140 includes a collet 142, a locking collar 170, and a locking bearing 180. As will be described in more detail herein, each of the locking collar 170 and the locking bearing 180 is slidingly disposed over the collet 142. Each of the locking collar 170 and the locking bearing 180 is configured to slide, translate, or move in a longitudinal direction by the user between at least a first or non-engaged position on the collet 142 and a second or engaged position on the collet 142.

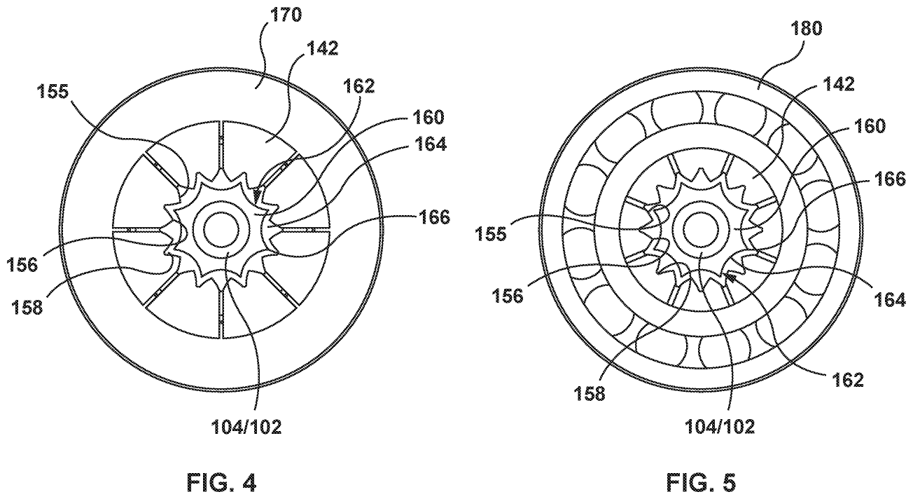

With reference to FIGS. 3-6, the collet 142 is a generally tubular element having a first end 144 and a second or opposing end 146. FIG. 4 is an end view of the locking mechanism 140 from the first end 144 of the collet 142, and FIG. 5 is an end view of the locking mechanism 140 from the second end 146 of the collet 142. A flange 145 is formed on the first end 144. The collet 142 has a first wall thickness T1 at the first end 144 and a second wall thickness T2 at the second end 146. The second wall thickness T2 is less than the first wall thickness T1. The wall thickness of the collet 142 gradually tapers from the first wall thickness T1 to the second wall thickness T2. As a result of the tapering wall thickness, the collet 142 has a first outer diameter D1 at the first end 144 and a second outer diameter D2 at the second end 146. The second outer diameter D2 is less than the first outer diameter D1. Approximately half or mid-way along the length of the collet 142, the collet 142 has a third outer diameter D3 that is less than the first outer diameter D1 and is greater than the second outer diameter D2.

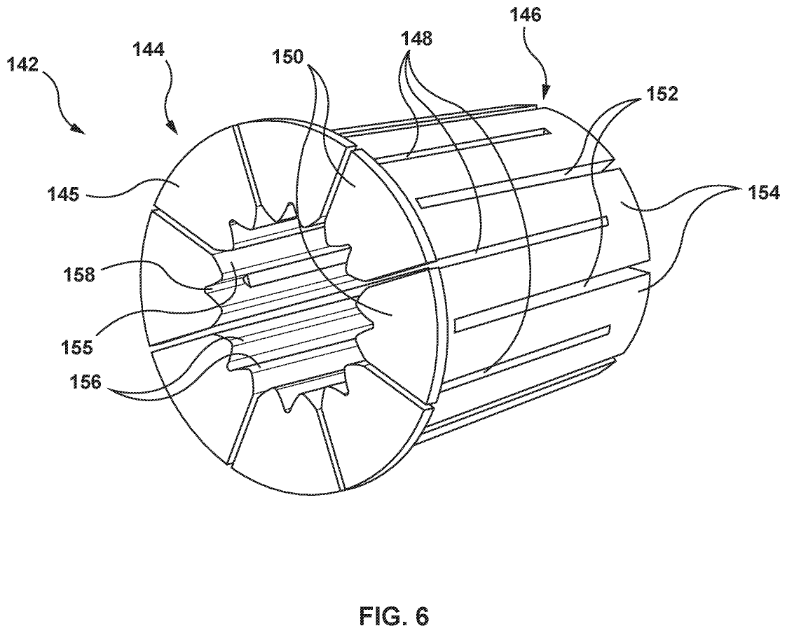

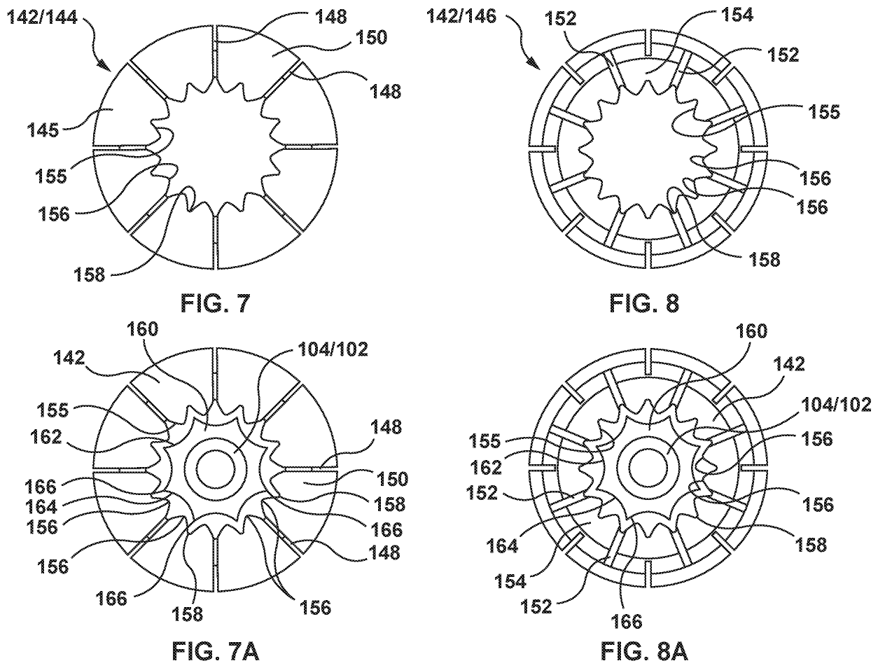

As best shown on FIGS. 6 and 7, the collet 142 includes a first plurality of slots 148 extending from the first end 144, towards the second end 146. FIG. 6 is a perspective view of the collet 142, and FIG. 7 is an end view of the collet 142 from the first end 144 of the collet 142. The first plurality of slots 148 do not extend to the second end 146, and therefore form a first plurality of integral tabs 150 that circumferentially spaced apart around the first end 144 of the collet 142. Each tab 150 of the first plurality of integral tabs 150 is defined by a pair of adjacent slots 148 of the first plurality of slots 148. The first plurality of slots 148 permit the first end 144 of the collet 142 to be radially compressible. When radially compressed, the width of the slots 148 decreases until adjacent tabs 150 of the first plurality of integral tabs 150 are wedged against each other.

As best shown in FIGS. 6 and 8, the collet 142 similarly includes a second plurality of slots 152 extending from the second end 146, towards the first end 144. FIG. 6 is a perspective view of the collet 142, and FIG. 8 is an end view of the collet 142 from the second end 146 of the collet 142. The second plurality of slots 152 do not extend to the first end 144, and therefore form a second plurality of integral tabs 154 that circumferentially spaced apart around the second end 146 of the collet 142. Each tab 154 of the second plurality of integral tabs 154 is defined by a pair of adjacent slots 152 of the second plurality of slots 152. The second plurality of slots 152 permit the second end 146 of the collet 142 to be radially compressible. When radially compressed, the width of the slots 152 decreases until adjacent tabs 154 of the second plurality of integral tabs 154 are wedged against each other. The first plurality of slots 148 are circumferentially offset from the second plurality of slots 152, with each slot 148 extending approximately half-way between a pair of adjacent slots 152 and each slot 152 extending approximately half-way between a pair of adjacent slots 148.

As shown in each of FIGS. 6, 7, and 8, the collet 142 includes an inner surface 155 with a sinusoidal configuration including a plurality of peaks 156 and a plurality of valleys 158. Each valley 158 is formed between a pair of adjacent peaks 156. Each tab 150 of the first plurality of integral tabs 150 includes two peaks 156 and a valley 158 formed therebetween. Similarly, each tab 154 of the second plurality of integral tabs 154 includes two peaks 156 and a valley 158 formed therebetween.

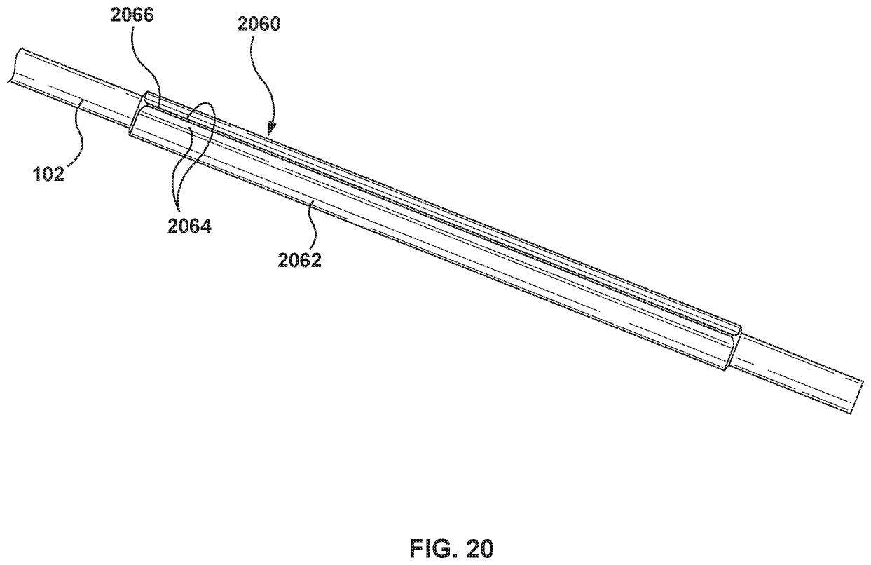

With reference to FIGS. 3, 4, 5, 7A, and 8A, the treatment catheter 102 includes a locking interface 160 on an outer surface thereof. In an embodiment, the locking interface 160 is formed as a separate component and is disposed on an outer surface of the treatment catheter 102 via any suitable mechanical method such as laser welding, heat welding, over-molding, adhesive binding, or other mechanical locking feature such as a threaded interface or a snap fit feature. The locking interface 160 is secured or fixed to the outer surface of the treatment catheter 102 so as to move as a single or unitary assembly. Stated another way, the locking interface 160 is fixed relative to the treatment catheter 102 such that they cannot move rotationally or axially relative to each other. In another embodiment hereof, the locking interface 160 may be integrally formed on an outer surface of the treatment catheter 102. The locking interface 160 has an outer surface 162 with a scalloped configuration including a plurality of grooves 164 and a plurality of ridges 166. Each groove 164 is formed between a pair of adjacent ridges 166. The collet 142 is disposed over the locking interface 160 and each groove 164 of the locking interface 160 is configured to receive a pair of adjacent peaks 156 of the collet 142. Stated another way, each groove 164 of the locking interface 160 is sized to receive two adjacent peaks 156 of the inner surface 155 of the collet 142. The scalloped configuration of the outer surface 162 of the locking interface is exemplary and the number of the plurality of grooves 164 and the plurality of ridges 166 may vary from that shown herein. For example, the locking interface 160 may include a fewer number of alternating ridges and grooves than shown, and the ridges and grooves are not required to be equally spaced around the outer surface 162 of the locking interface 160. The number of grooves 164 are not required to match or equal the number of ridges 166. Alternative embodiments of the scalloped configuration are described in more detail herein with respect to FIGS. 20-22.

As best shown on FIG. 7A, which is an end view of the first end 144 of the collet 142 and the locking interface 160 disposed therein, at the first end 144 of the collet 142, each ridge 166 of the locking interface 160 aligns with a valley 158 of the inner surface 155 of the collet 142. Further, at the first end 144 of the collet 142, each slot 148 of the first plurality of slots 148 extends between the two adjacent peaks 156 that are received within the groove 164 of the locking interface 160.

As best shown on FIG. 8A, which is an end view of the second end 146 of the collet 142 and the locking interface 160 disposed therein, at the second end 146 of the collet 142, each ridge 166 of the locking interface 160 aligns with a slot 152 of the second plurality of slots 152. Further, at the second end 146 of the collet 142, a valley 158 extends between the two adjacent peaks 156 that are received within the groove 164 of the locking interface 160.

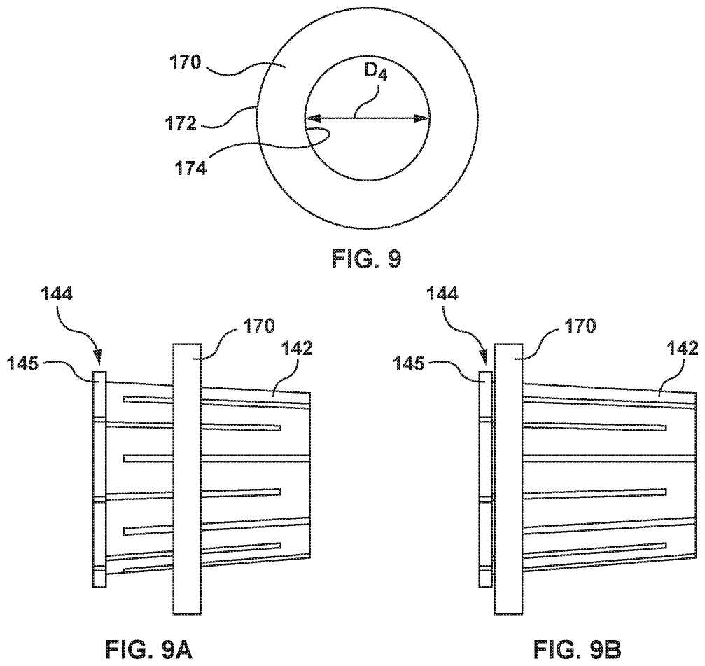

With additional reference to FIG. 9, the locking collar 170 is an annular or ring element that is slidingly disposed over the outer surface of the collet 142, and may be moved or translated in a longitudinal direction by the user. The locking collar 170 is accessible to the user via an actuator 117, which may be a slider, switch, or any suitable mechanical device that extends through the housing 132 and is externally accessible such that the locking collar 170 may be manipulated or moved by the user. An inner surface 174 of the locking collar 170 is sized or configured to have a minimum inner diameter D4, which is equal to or substantially similar to the third outer diameter D3 of the collet 142 and is less than the first outer diameter D1 of the collet 142. In an embodiment, as best shown in FIG. 3A, the inner surface 174 of the locking collar 170 is angled or tapered to correspond or mate with the tapered outer surface of the collet 142. In another embodiment, the inner surface 174 of the locking collar is not angled or tapered but rather has a cylindrical inner surface with the collet 142 having a tapered outer surface such that there is a point of contact between the sliding surfaces. In the first or non-engaged position on the collet 142, as shown on FIG. 9A, the locking collar 170 is disposed over a midportion of the collet 142. In the first or non-engaged position on the collet 142, the inner surface 174 of the locking collar 170 may contact the outer surface of the collet 142 but the locking collar 170 does not radially compress the collet 142 since the minimum inner diameter D4 of the locking collar 170 is equal to or substantially similar to the third outer diameter D3 of the collet 142 at the midportion of the collet 142. In the second or engaged position on the collet 142, which is shown on FIG. 9B, the locking collar 170 is disposed adjacent to or near the first end 144 of the collet 142. In the second or engaged position on the collet 142, the inner surface 174 of the locking collar 170 contacts the outer surface of the collet 142 and the locking collar 170 radially compresses the collet 142 since the minimum inner diameter D4 of the locking collar 170 is less than the first outer diameter D1 of the collet 142 at the first end 144 thereof. Thus, as the locking collar 170 is moved from the first or non-engaged position on the collet 142 towards the second or engaged position on the collet 142, the locking collar 170 radially compresses the first end 144 of the collet 142.

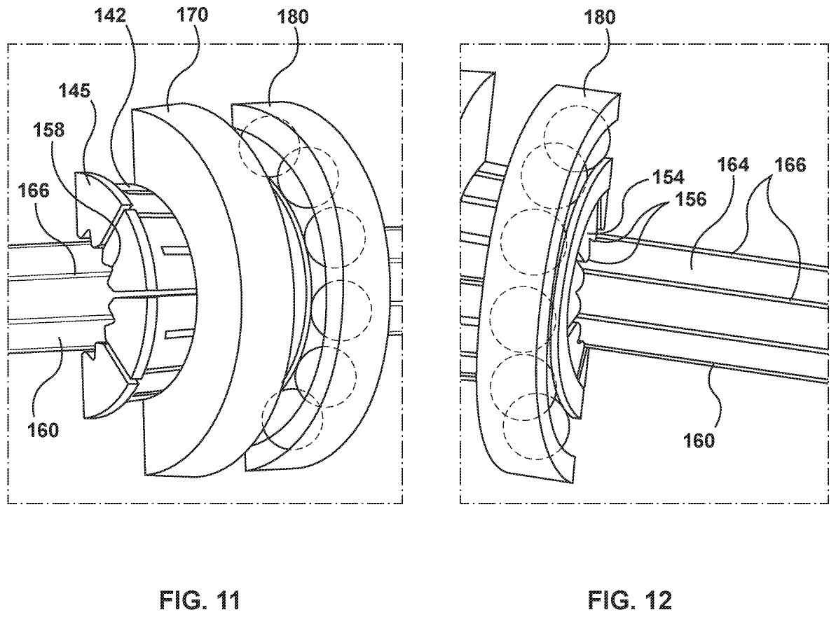

When the locking collar 170 is in the second or engaged position on the collet 142, the locking collar 170 radially compresses the collet 142 and thereby prevents or restricts rotation of the treatment catheter 102. As best shown in the enlarged perspective view of FIG. 11, when the first end 144 of the collet 142 is radially compressed by the locking collar 170, each ridge 166 of the locking interface 160 extends into or is received within a valley 158 of the inner surface 155 of the collet 142. With the ridges 166 of the locking interface 160 protruding into valleys 158 of the collet 142, the locking interface 160 and treatment catheter 102 attached thereto is prevented from rotating. Thus, when the locking collar 170 is in the second or engaged position on the collet 142, the treatment catheter 102 is no longer permitted to be rotated relative to the guide catheter 102. However, when the locking collar 170 is in the second or engaged position on the collet 142, the locking collar 170 still permits axial translation of the treatment catheter 102 because the ridges 166 of the locking interface 160 are permitted to slide within the valleys 158 of the collet 142.

Notably, although the locking collar 170 is described herein as having only two positions, i.e., a first or non-engaged position and a second or engaged position, it will be understood that the locking collar 170 has numerous positions intermediate or in between the first and second positions. As the locking collar 170 is advanced from the midportion of the collet 142 towards the first end 144 of the collet 142, over the tapered outer surface of the collet 142, gradual radial compression of the collet 142 occurs. As used herein, the first or non-engaged position includes a range of positions of the locking collar 170 relative to the collet 142 in which the locking collar 170 does not radially compress the collet 142 to the extent by which rotation of the treatment catheter 102 is prevented. Conversely, as used herein, the second or engaged position includes a range of positions of the locking collar 170 relative to the collet 142 in which the locking collar 170 radially compresses the collet 142 to the extent by which rotation of the treatment catheter 102 is prevented.

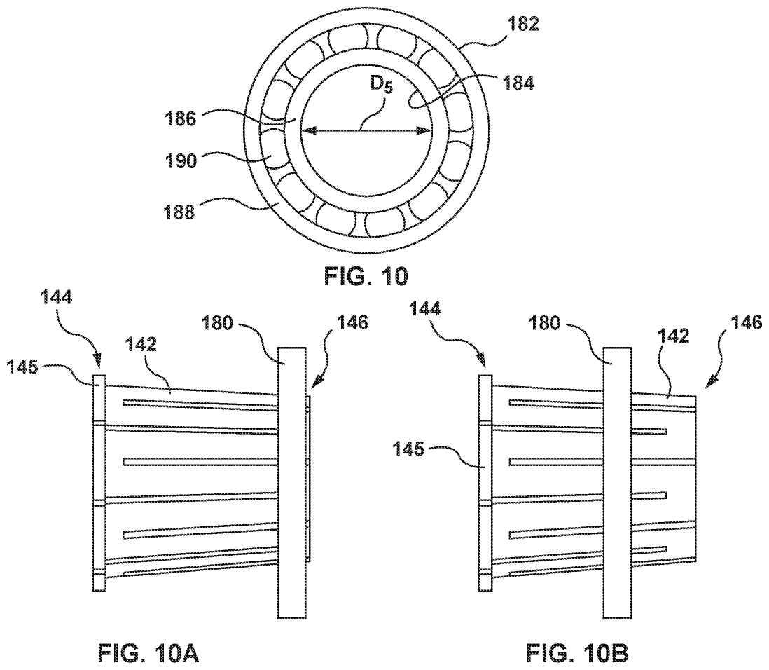

With additional reference to FIG. 10, the locking bearing 180 is a ball bearing that is slidingly disposed over the outer surface of the collet 142, and may be moved or translated in a longitudinal direction by the user. The locking bearing 180 is accessible to the user via an actuator 121, which may be a slider, switch, or any suitable mechanical device that extends through the housing 132 and is externally accessible such that the locking bearing 180 may be manipulated or moved by the user. An inner surface 184 of the locking bearing 180 is sized or configured to have a minimum inner diameter D5, which is equal to or slightly greater than the second outer diameter D2 of the collet 142 and is less than the third outer diameter D3 of the collet 142. In an embodiment, as best shown in FIG. 3A, the inner surface 184 of the locking bearing 180 is angled or tapered to correspond or mate with the tapered outer surface of the collet 142. In another embodiment, the inner surface 184 of the locking bearing is not angled or tapered but rather has a cylindrical inner surface with the collet 142 having a tapered outer surface such that there is a point of contact between the sliding surfaces. The locking bearing 180 includes an inner bearing race 186, an outer bearing race 188, and a plurality of balls 190 disposed between the inner and outer bearing races 186, 188. The inner and outer bearing races 186, 188 are concentric annular or ring components, and the inner bearing race 186 is disposed or positioned within the outer bearing race 188. When one of the inner bearing race 186 and the outer bearing race 188 rotates, the rotation thereof causes the balls 190 to rotate as well while the non-rotating bearing race stays stationary. For example, rotation of the inner bearing race 186 causes the balls 190 to rotate as well, such that the inner bearing race 186 and the balls 190 rotate relative to the outer bearing race 188.

In the first or non-engaged position on the collet 142, which is shown in FIG. 10A, the locking bearing 180 is disposed over the second end 146 of the collet 142. In the first or non-engaged position on the collet 142, the inner surface 184 of the locking bearing 180 may contact the outer surface of the collet 142 but the locking bearing 180 does not radially compress the collet 142 since the minimum inner diameter D5 of the locking bearing 180 is equal to or slightly greater than the second outer diameter D2 of the collet 142 at the second end 146 of the collet 142. In the second or engaged position on the collet 142, the locking bearing 180 is disposed adjacent to or near the midportion of the collet 142. In the second or engaged position on the collet 142, which is shown in FIG. 10B, the inner surface 184 of the locking bearing 180 contacts the outer surface of the collet 142 and the locking bearing 180 radially compresses the collet 142 since the minimum inner diameter D5 of the locking bearing 180 is less than the third outer diameter D2 of the collet 142 at the midportion thereof. Thus, as the locking bearing 180 is moved from the first or non-engaged position on the collet 142 towards the second or engaged position on the collet 142, the locking bearing 180 radially compresses the midportion of the collet 142.

When the locking bearing 180 is in the second or engaged position on the collet 142, the locking bearing 180 radially compresses the collet 142 and thereby prevents or restricts axial translation of the treatment catheter 102. As best shown on the enlarged perspective view of FIG. 12, when the midportion of the collet 142 is radially compressed by the locking bearing 180, each tab 154 of the second plurality of tabs 154 are wedged into a groove 164 of the locking interface 160. Each tab 154 of the second plurality of tabs 154 includes two adjacent peaks 156 and a valley 158 therebetween, with two slots 152 on either side of the tab 154. With slots 152 on either side of each tab 154, the tabs 154 are pushed into the grooves 164 of the locking interface 160 and the midportion of the collet 142 is radially compressed by the locking bearing 180. With the tabs 154 wedged into the grooves 164, the locking interface 160 and treatment catheter 102 attached thereto cannot move axially, or are not permitted to move axially, under normal operating conditions. Thus, when the locking bearing 180 is in the second or engaged position on the collet 142, the treatment catheter 102 is no longer permitted to be axially translated or moved in a longitudinal direction relative to the guide catheter 102.

However, when the locking bearing 180 is in the second or engaged position on the collet 142, the locking bearing 180 still permits rotation of the treatment catheter 102 because the inner bearing race 186 rotates relative to the outer bearing race 188. More particularly, with the tabs 154 of the collet 142 wedged into the grooves 164 of the locking interface 160, the collet 142 is mechanically interlocked with the locking interface 160. When a user rotates the treatment catheter 102 (thus rotating the locking interface 160 attached thereto), the assembly of the collet 142 and the inner bearing race 186 rotates therewith. Although the locking bearing 180 is radially compressing the midportion of the collet 142, the locking bearing 180 still permits rotation of the collet since the locking bearing 180 is a ball bearing. The inner bearing race 186 and balls 190 rotate with the collet 142, while outer bearing race 188 stays stationary.

Notably, although the locking bearing 180 is described herein as having only two positions, i.e., a first or non-engaged position and a second or engaged position, it will be understood that the locking bearing 180 has numerous positions intermediate or in between the first and second positions. As the locking bearing 180 is advanced from the second end 146 towards the midportion of the collet 142, over the tapered outer surface of the collet 142, gradual radial compression of the collet 142 occurs. As used herein, the first or non-engaged position includes a range of positions of the locking bearing 180 relative to the collet 142 in which the locking bearing 180 does not radially compress the collet 142 to the extent by which axial translation of the treatment catheter 102 is prevented. Conversely, as used herein, the second or engaged position includes a range of positions of the locking bearing 180 relative to the collet 142 in which the locking bearing 180 radially compresses the collet 142 to the extent by which axial translation of the treatment catheter 102 is prevented.

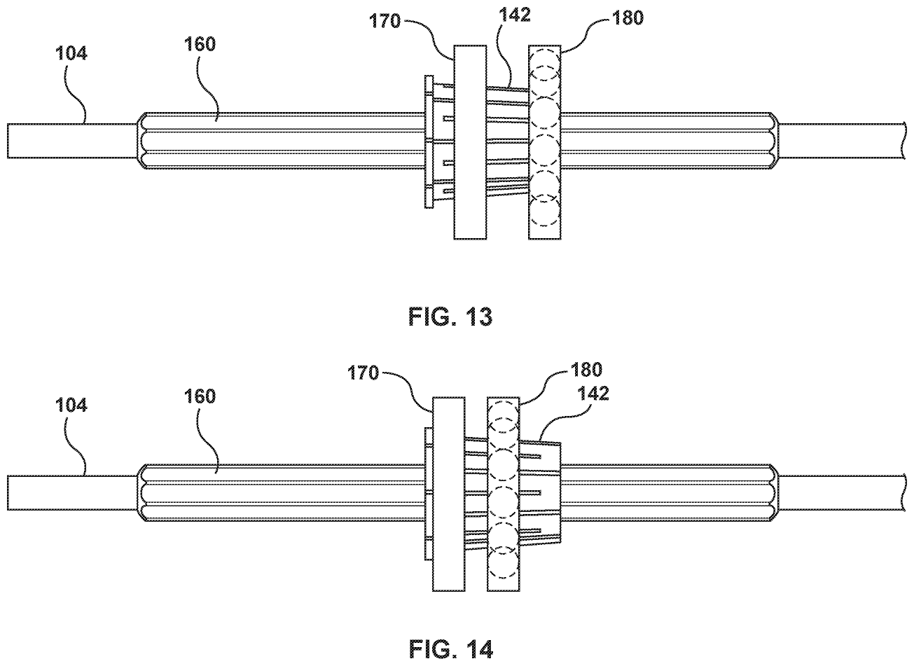

The four states or configurations of the locking mechanism 140 (a locked state, an unlocked state, a first semi-locked state, and a second semi-locked state) are described in turn in more detail below. As described above, the locking mechanism 140 includes an unlocked state or configuration in which the treatment catheter 102 is permitted to slide freely in a longitudinal direction relative to the guide catheter 102 and is permitted to rotate freely in a circumferential direction relative to the guide catheter 102. In the unlocked state or configuration, which is shown in FIG. 13, each of the locking collar 170 and the locking bearing 180 is disposed in the first or non-engaged position on the collet 142. More particularly, in the first or non-engaged position on the collet 142, the locking collar 170 is disposed over a midportion of the collet 142 and the locking bearing 180 is disposed over the second end 146 of the collet 142. In the first or non-engaged position on the collet 142, neither the locking collar 170 nor the locking bearing 180 radially compresses the collet 142. Thus, in the unlocked state or configuration of the locking mechanism 140, the collet 142 does not interact with the locking interface 160 of the treatment catheter 102 and the treatment catheter 102 may be axially translated and/or rotated relative to the collet 142 by user manipulation of the treatment catheter 102. For example, a user can axially translation or rotate the treatment catheter 102 via manipulation of bifurcated luer 116 which extends proximally from the handle 130. Each of the locking collar 170 and the locking bearing 180 contacts and abuts against the collet 142 when in their respective first or non-engaged position in order to maintain or hold the collet 142 centrally around the locking collar 170 and prevent any inadvertent movement thereof. Stated another way, the locking collar 170 and the locking bearing 180 in their respective first or non-engaged positions maintain or hold the collet 142 in a radially spaced position relative to the locking interface 160.

The locking mechanism 140 also includes a locked state or configuration in which the treatment catheter 102 is not permitted to slide freely in the longitudinal direction relative to the guide catheter 120 and is not permitted to rotate freely in the circumferential direction relative to the guide catheter 120. In the locked state or configuration, which is shown in FIG. 14, each of the locking collar 170 and the locking bearing 180 is disposed in the second or engaged position on the collet 142. More particularly, in the second or engaged position on the collet 142, the locking collar 170 is disposed over the first end 144 of the collet 142 and the locking bearing 180 is disposed over the midportion of the collet 142. In the second or engaged position on the collet 142, both the locking collar 170 and the locking bearing 180 radially compresses the collet 142.

More particularly, when the locking collar 170 is in the second or engaged position on the collet 142, the locking collar 170 radially compresses the collet 142 and thereby prevents or restricts rotation of the treatment catheter 102. When the first end 144 of the collet 142 is radially compressed by the locking collar 170, each ridge 166 of the locking interface 160 extends into or is received within a valley 158 of the inner surface 155 of the collet 142. With the ridges 166 of the locking interface 160 protruding into valleys 158 of the collet 142, the locking interface 160 and treatment catheter 102 attached thereto is prevented from rotating. Thus, when the locking collar 170 is in the second or engaged position on the collet 142, the treatment catheter 102 is no longer permitted to be rotated relative to the guide catheter 102.

When the locking bearing 180 is in the second or engaged position on the collet 142, the locking bearing 180 radially compresses the collet 142 and thereby prevents or restricts axial translation of the treatment catheter 102. When the midportion of the collet 142 is radially compressed by the locking bearing 180, each tab 154 of the second plurality of tabs 154 are wedged into a groove 164 of the locking interface 160. Each tab 154 of the second plurality of tabs 154 includes two adjacent peaks 156 and a valley 158 therebetween, with two slots 152 on either side of the tab 154. With slots 152 on either side of each tab 154, the tabs 154 are pushed into the grooves 164 of the locking interface 160 the midportion of the collet 142 is radially compressed by the locking bearing 180. With the tabs 154 wedged into the grooves 164, the locking interface 160 and treatment catheter 102 attached thereto cannot move axially, or are not permitted to move axially, under normal operating conditions. Thus, when the locking bearing 180 is in the second or engaged position on the collet 142, the treatment catheter 102 is no longer permitted to be axially translated or moved in a longitudinal direction relative to the guide catheter 102.

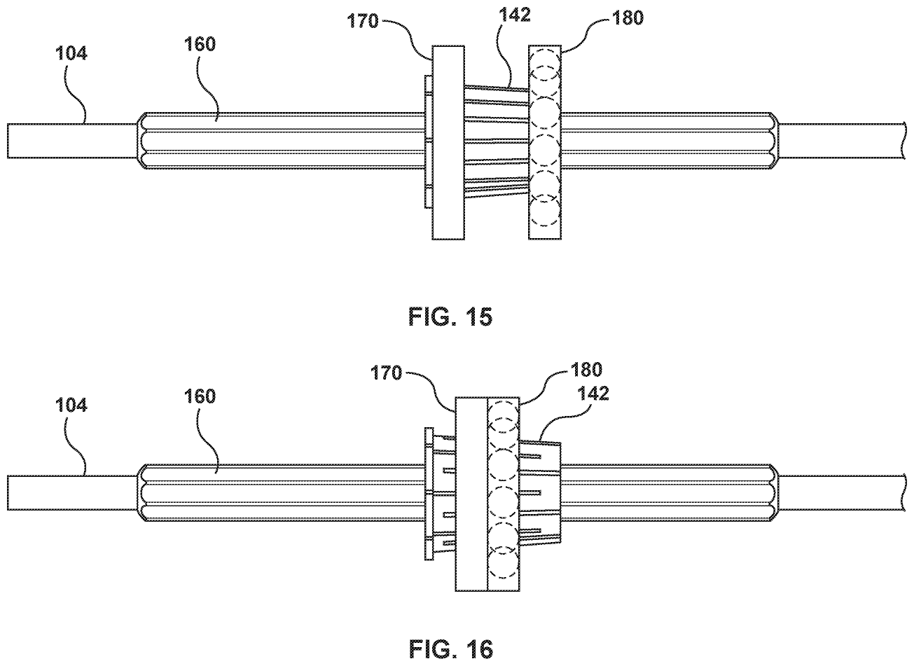

The locking mechanism 140 also includes a first semi-locked state or configuration in which the treatment catheter 102 is permitted to slide freely in a longitudinal direction relative to the guide catheter 120 and is not permitted to rotate freely in a circumferential direction relative to the guide catheter 120, thereby restricting the treatment catheter 102 to only longitudinal movement in the proximal or distal direction while preventing inadvertent rotation of the treatment catheter 102. In the first semi-locked state or configuration, which is shown in FIG. 15, the locking collar 170 is disposed in the second or engaged position and the locking bearing 180 is disposed in the first or non-engaged position on the collet. More particularly, the locking collar 170 is disposed over the first end 144 of the collet 142 and the locking bearing 180 is disposed over the second end 146 of the collet 142.

In the first or non-engaged position on the collet 142, the locking bearing 180 is disposed over the second end 146 of the collet 142 and does not radially compress the collet 142. Thus, when the locking bearing 180 is in the first or non-engaged position on the collet 142, the locking bearing 180 is not causing the collet 142 to restrict or prevent any movement of the treatment catheter 102. Stated another way, with the locking bearing 180 at the second end 146 of the collet 142, the second end 146 of the collet 142 does not interact with the locking interface 160 of the treatment catheter 102 and the treatment catheter 102 may be axially translated and/or rotated relative to the collet 142 by user manipulation of the treatment catheter 102.

When the locking collar 170 is in the second or engaged position on the collet 142, the locking collar 170 radially compresses the first end 144 of the collet 142 and thereby prevents or restricts rotation of the treatment catheter 102. When the first end 144 of the collet 142 is radially compressed by the locking collar 170, each ridge 166 of the locking interface 160 extends into or is received within a valley 158 of the inner surface 155 of the collet 142. With the ridges 166 of the locking interface 160 protruding into valleys 158 of the collet 142, the locking interface 160 and treatment catheter 102 attached thereto is prevented from rotating. Thus, when the locking collar 170 is in the second or engaged position on the collet 142, the treatment catheter 102 is no longer permitted to be rotated relative to the guide catheter 102. However, when the locking collar 170 is in the second or engaged position on the collet 142, the locking collar 170 still permits axial translation of the treatment catheter 102 because the ridges 166 of the locking interface 160 are permitted to slide within the valleys 158 of the collet 142. Stated another way, the degree or amount of radial compression required for the locking collar 170 to be in the second or engaged position (and thereby prevent rotation of the treatment catheter 102) does not interfere with axial translation of the treatment catheter 102. The degree or amount radial compression is sufficient to ensure each ridge 166 of the locking interface 160 extends into or is received within a valley 158 of the inner surface 155 of the collet 142 to prevent rotation, but the locking collar 170 does not compress the collet 142 to the extent which would prevent axial translation.

The locking mechanism 140 also includes a second semi-locked state or configuration in which the treatment catheter 102 is not permitted to slide freely in a longitudinal direction relative to the guide catheter 120 and is permitted to rotate freely in a circumferential direction relative to the guide catheter 120, thereby permitting torqueing or rotation of the treatment catheter 102 while preventing inadvertent translation of the treatment catheter 102 in the proximal or distal direction. In the second semi-locked state or configuration, which is shown in FIG. 16, the locking collar 170 is disposed in the first or non-engaged position on the collet 142 and the locking bearing 180 is disposed in the second or engaged position on the collet. More particularly, each of the locking collar 170 and the locking bearing 180 is disposed over the midportion of the collet 142.

In the first or non-engaged position on the collet 142, the locking collar 170 is disposed over the midportion of the collet 142 and does not radially compress the collet 142. Thus, when the locking collar 170 is in the first or non-engaged position on the collet 142, the locking collar 170 is not causing the collet 142 to restrict or prevent any movement of the treatment catheter 102. Stated another way, with the locking collar 170 at the midportion of the collet 142, the locking collar 170 is not causing the midportion of the collet 142 to interact with the locking interface 160 of the treatment catheter 102 and the treatment catheter 102 may be axially translated and/or rotated relative to the collet 142 by user manipulation of the treatment catheter 102.

When the locking bearing 180 is in the second or engaged position on the collet 142, the locking bearing 180 radially compresses the midportion of the collet 142 and thereby prevents or restricts axial translation of the treatment catheter 102. When the midportion of the collet 142 is radially compressed by the locking bearing 180, each tab 154 of the second plurality of tabs 154 are wedged into a groove 164 of the locking interface 160. Each tab 154 of the second plurality of tabs 154 includes two adjacent peaks 156 and a valley 158 therebetween, with two slots 152 on either side of the tab 154. With slots 152 on either side of each tab 154, the tabs 154 are pushed into the grooves 164 of the locking interface 160 the midportion of the collet 142 is radially compressed by the locking bearing 180. With the tabs 154 wedged into the grooves 164, the locking interface 160 and treatment catheter 102 attached thereto cannot move axially, or are not permitted to move axially, under normal operating conditions. Thus, when the locking bearing 180 is in the second or engaged position on the collet 142, the treatment catheter 102 is no longer permitted to be axially translated or moved in a longitudinal direction relative to the guide catheter 102. However, when the locking bearing 180 is in the second or engaged position on the collet 142, the locking bearing 180 still permits rotation of the treatment catheter 102 because the inner bearing race 186 rotates relative to the outer bearing race 188. More particularly, with the tabs 154 of the collet 142 wedged into the grooves 164 of the locking interface 160, the collet 142 is mechanically interlocked with the locking interface 160. When a user rotates the treatment catheter 102 (thus rotating the locking interface 160 attached thereto), the assembly of the collet 142 and the inner bearing race 186 rotates therewith. Although the locking bearing 180 is radially compressing the midportion of the collet 142, the locking bearing 180 still permits rotation of the collet since the locking bearing 180 is a ball bearing. The inner bearing race 186 and balls 190 rotate with the collet 142, while outer bearing race 188 stays stationary.

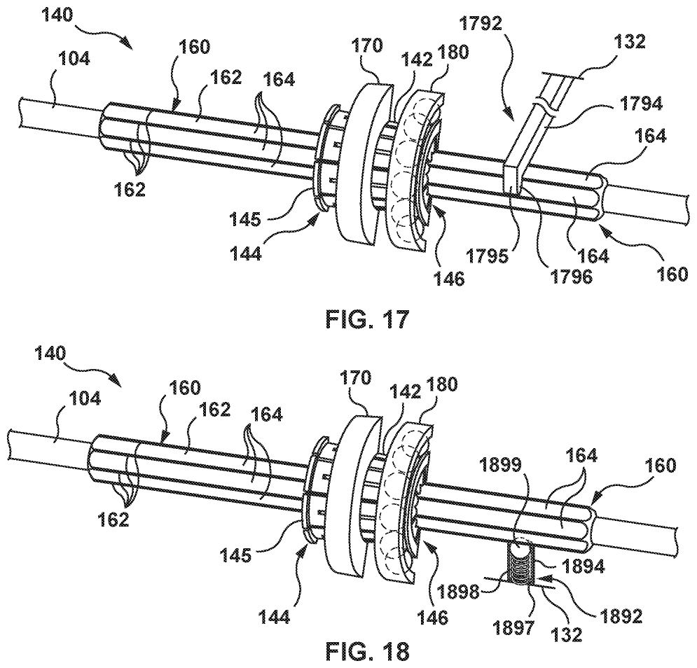

The handle 130 may also include an indexing component configured to output a tactile click at one or more defined degrees of rotation of the treatment catheter 102. The indexing feature tracks user rotation of the treatment catheter 102 in defined increments. An exemplary indexing component 1792 that may be utilized in embodiments hereof is depicted in FIG. 17. In the embodiment of FIG. 17, the indexing component 1792 includes an indexing finger or lever 1794. FIG. 17 illustrates the locking mechanism 140 and the indexing component 1792 removed from the housing 132 of the handle 130 for sake of illustration. When assembled into the housing 132, the indexing finger 1794 is disposed within the handle 130, and includes a first end (not shown) attached to an interior surface of the housing 132 of the handle 130 and a second end that is unattached or free. The second end includes a radially-extending protrusion 1795 with a pointed tip 1796. The indexing finger 1794 is configured to interact with the locking interface 160 to track rotation of the treatment catheter 102. More particularly, the pointed tip 1796 of the indexing finger 1794 is sized and configured to be received within a single groove 164 of the locking interface 160. The pointed tip 1796 moves to an adjacent groove 164 when the treatment catheter 102 and locking interface 160 attached thereto rotates. In an embodiment in which the locking interface 160 includes a total of six grooves 164, movement between each groove indicates rotation of 60 degrees. Stated another way, when the locking interface 160 includes a total of six grooves 164, the indexing finger 1794 tracks rotation of the treatment catheter 102 in stepped increments of 60 degrees each. It will be apparent to one of ordinary skill in the art that the locking interface 160 may be modified to include a greater or lesser number of grooves, and a greater number of grooves results in smaller stepped increments and a lesser number of grooves results in larger stepped increments. For example, if the locking interface is modified to include a total of 12 grooves, movement between each groove would indicate rotation of 30 degrees. A tactile click is output each time the indexing finger 1794 moves into a new groove 164 in order to provide tactile feedback of rotation to the user.

The indexing component 1792 may be utilized to track user rotation of the treatment catheter 102 while the locking mechanism 140 is in the an unlocked state or configuration, as well as when the locking mechanism is in the second semi-locked state or configuration. As described above with respect to FIG. 13, in the unlocked state or configuration, the treatment catheter 102 is permitted to slide freely in a longitudinal direction relative to the guide catheter 102 and is permitted to rotate freely in a circumferential direction relative to the guide catheter 102. Further, as described above with respect to FIG. 16, in the second semi-locked state or configuration, the treatment catheter 102 is not permitted to slide freely in a longitudinal direction relative to the guide catheter 120 and is permitted to rotate freely in a circumferential direction relative to the guide catheter 120, thereby permitting torqueing or rotation of the treatment catheter 102 while preventing inadvertent translation of the treatment catheter 102 in the proximal or distal direction. Since the indexing component 1792 is configured to interact with the locking interface 160, the indexing component 1792 tracks user rotation of the treatment catheter 102 when the locking mechanism 140 is in either of these configurations.

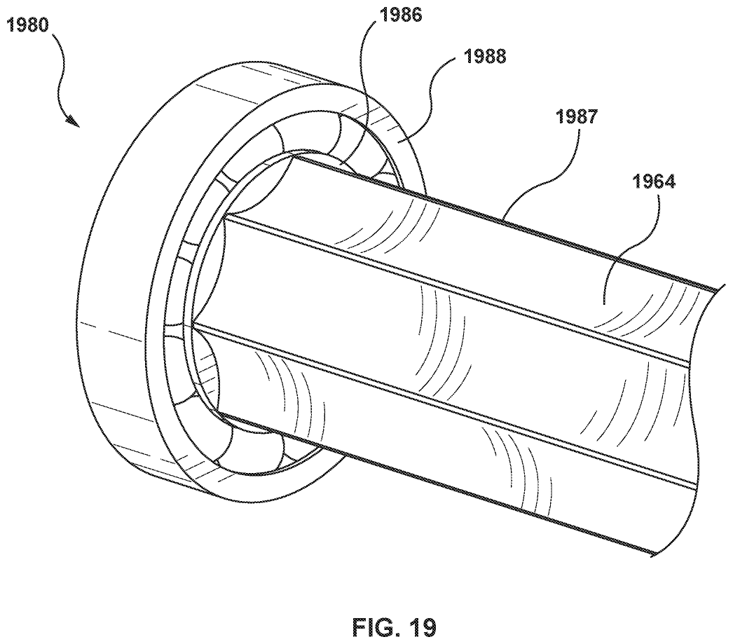

Another exemplary indexing component 1892 that may be utilized in embodiments hereof is depicted in FIG. 18. In the embodiment of FIG. 18, the indexing component 1792 includes a spring loaded ball bearing 1894. The spring loaded ball bearing 1894 includes a housing 1897, a spring 1898 disposed within the housing 1897, and a sphere or ball 1899 partially disposed within the housing 1897. The spring 1898 biases the sphere 1899 into an extended configuration or position, but permits the sphere 1899 to move within the housing 1897 when sufficient force is applied thereto. FIG. 18 illustrates the locking mechanism 140 and the indexing component 1892 removed from the housing 132 of the handle 130 for sake of illustration. When assembled into the housing 132, the spring loaded ball bearing 1894 is disposed within the handle 130, and includes a first end attached to an interior surface of the housing 132 of the handle 130 and a second end that is unattached or free. The second end includes the sphere 1899. The sphere 1899 is configured to interact with the locking interface 160 to track rotation of the treatment catheter 102. More particularly, the sphere 1899 of the spring loaded ball bearing 1894 is sized and configured to be received within a single groove 164 of the locking interface 160. The sphere 1899 moves to an adjacent groove 164 when the treatment catheter 102 and locking interface 160 attached thereto rotates. When the treatment catheter 102 and locking interface 160 attached thereto is rotated, the sphere 1899 is pushed or moved into the housing 1897 due to longitudinal compression of the spring 1898. Once the sphere 1899 is aligned with a groove 164, the spring 1898 resumes its initial configuration and pushes or advanced the sphere 1899 back to the extended configuration in which the sphere 1899 is received within the groove 164. In an embodiment in which the locking interface 160 includes a total of six grooves 164, movement between each groove indicates rotation of 60 degrees. Stated another way, when the locking interface 160 includes a total of six grooves 164, the spring loaded ball bearing 1894 tracks rotation of the treatment catheter 102 in stepped increments of 60 degrees each. It will be apparent to one of ordinary skill in the art that the locking interface 160 may be modified to include a greater or lesser number of grooves, and a greater number of grooves results in smaller stepped increments and a lesser number of grooves results in larger stepped increments. For example, if the locking interface is modified to include a total of 12 grooves, movement between each groove would indicate rotation of 30 degrees. A tactile click is output each time the sphere 1899 moves into a new groove 164 in order to provide tactile feedback of rotation to the user.

The indexing component 1892 may be utilized to track user rotation of the treatment catheter 102 while the locking mechanism 140 is in the an unlocked state or configuration, as well as when the locking mechanism is in the second semi-locked state or configuration. As described above with respect to FIG. 13, in the unlocked state or configuration, the treatment catheter 102 is permitted to slide freely in a longitudinal direction relative to the guide catheter 102 and is permitted to rotate freely in a circumferential direction relative to the guide catheter 102. Further, as described above with respect to FIG. 16, in the second semi-locked state or configuration, the treatment catheter 102 is not permitted to slide freely in a longitudinal direction relative to the guide catheter 120 and is permitted to rotate freely in a circumferential direction relative to the guide catheter 120, thereby permitting torqueing or rotation of the treatment catheter 102 while preventing inadvertent translation of the treatment catheter 102 in the proximal or distal direction. Since the indexing component 1892 is configured to interact with the locking interface 160, the indexing component 1792 tracks user rotation of the treatment catheter 102 when the locking mechanism 140 is in either of these configurations.

Although indexing components 1794, 1894 are described above as being configured to interact with the grooves 164 of the locking interface 160, the indexing components may alternatively be configured to interact with the locking bearing 180. More particularly, with reference to FIG. 19, a locking bearing 1980 is shown that is configured to interact with indexing components 1794, 1894. The locking bearing 1980 includes an inner bearing race 1986 that includes an extension which is longer or extended relative to an outer bearing race 1988 thereof. The extension may be integral to or attached to the inner bearing race 1986. When assembled over the treatment catheter 102, the extension of the inner bearing race 1986 would be coaxially disposed over the locking interface 160 thereof (not shown in FIG. 19). The extension of the inner bearing race 1986 provides an outer or exposed surface 1987 that includes a plurality of grooves 1964 formed thereon that are configured to interact with the indexing components 1794, 1894 in a similar manner as the grooves 164 of the locking interface 160. In this embodiment, however, the number of grooves 1964 on the extension of the inner bearing race 1986 may be solely chosen to optimize the increments for tracking rotation of the treatment catheter 102. For example, in an embodiment hereof, in which the outer surface 1987 includes a total of thirty-six grooves 1964, movement between each groove indicates rotation of 10 degrees. Stated another way, when the extension of the inner bearing race 1986 includes a total of thirty-six grooves 1964 on the outer surface 1987 thereof, the indexing component 1794, 1894 tracks rotation of the treatment catheter 102 in stepped increments of 10 degrees each. It will be apparent to one of ordinary skill in the art that the extension of the inner bearing race 1986 may be modified to include a greater or lesser number of grooves, and a greater number of grooves results in smaller stepped increments and a lesser number of grooves results in larger stepped increments. For example, if the outer surface of the extension of the inner bearing race is modified to include a total of 18 grooves, movement between each groove would indicate rotation of 20 degrees.

In the embodiment of FIG. 19, the indexing component 1792, 1892 may be utilized to track user rotation of the treatment catheter 102 while the locking mechanism 140 is in the second semi-locked state or configuration only. As described above with respect to FIG. 16, in the second semi-locked state or configuration, the treatment catheter 102 is not permitted to slide freely in a longitudinal direction relative to the guide catheter 120 and is permitted to rotate freely in a circumferential direction relative to the guide catheter 120, thereby permitting torqueing or rotation of the treatment catheter 102 while preventing inadvertent translation of the treatment catheter 102 in the proximal or distal direction. Since the indexing component 1792, 1892 is configured to interact with the locking bearing 1980, the indexing component 1792, 1892 tracks user rotation of the treatment catheter 102 only when the inner bearing race 1986 is engaged and rotated via rotation of the treatment catheter 102.

Although the locking mechanism 140 described herein includes a total of four states or configurations including a locked state, an unlocked state, a first semi-locked state, and a second semi-locked state, the locking mechanism 140 may be modified to permit various combinations of the above-referenced states. For example, in another embodiment hereof, the locking mechanism 140 may be modified such that only the locking collar 170 is permitted to switch between the engaged and non-engaged positions, while the locking bearing 180 is disposed at the engaged position. In such an embodiment, the locking mechanism would have only two states or configurations: (1) a first position in which the locking collar 170 is in the engaged position and neither longitudinal nor rotational movement of the treatment catheter 102 is permitted, and (2) a second position in which the locking collar 170 is in the non-engaged position and the locking mechanism allows the treatment catheter 102 to rotate relative to the guide catheter 120 but does not allow for longitudinal movement of the treatment catheter 102. In such an embodiment, the locking collar 170 essentially functions as a switch which when toggled can permit or prevent rotation of the treatment catheter 102 relative to the guide catheter 120. This embodiment may be desirable if the user has already located the balloon-expandable prosthesis 110 longitudinally and does not want the prosthesis to move longitudinally when rotated or torqued.

In another embodiment hereof, the locking mechanism 140 may be modified such that only the locking bearing 180 is permitted to switch between the engaged and non-engaged positions, while the locking collar 170 is disposed at the engaged position. In such an embodiment, the locking mechanism would have only two states or configurations: (1) a first position in which the locking bearing 180 is in the engaged position and neither longitudinal nor rotational movement of the treatment catheter 102 is permitted, and (2) a second position in which the locking bearing 180 is in the non-engaged position and the locking mechanism allows the treatment catheter 102 to move longitudinally relative to the guide catheter 120 but does not allow for rotation of the treatment catheter 102. In such an embodiment, the locking bearing 180 essentially functions as a switch which when toggled can permit or prevent axial translation of the treatment catheter 102 relative to the guide catheter 120.

In another embodiment hereof, the locking mechanism 140 may be modified such that only the locking bearing 180 is permitted to switch between the engaged and non-engaged positions, while the locking collar 170 is disposed at the non-engaged position. In such an embodiment, the locking mechanism would have only two states or configurations: (1) a first position in which the locking bearing 180 is in the engaged position and only rotational movement of the treatment catheter 102 is permitted, but does not allow for axial translation of the treatment catheter 102, and (2) a second position in which the locking bearing 180 is in the non-engaged position and the locking mechanism allows for both rotational and longitudinal movement of the treatment catheter 102. In such an embodiment, the locking bearing 180 essentially functions as a switch which when toggled can permit or prevent axial translation of the treatment catheter 102 relative to the guide catheter 120.

The locking mechanism 140 is described herein for controlling relative movement between the treatment catheter 102 and the guide catheter 120. However, the locking mechanism 140 may be utilized for controlling relative movement between any two catheter-type devices. The treatment catheter 102 is not required to be a balloon catheter but rather can any treatment or diagnostic catheter device, including for example, a stent delivery catheter, a drug delivery catheter, or an imaging catheter. The treatment catheter 102 may, for example, be a diagnostic catheter for measuring one or more hemodynamic conditions including ejection fraction, pressure differential, aortic jet velocity, or doppler velocity index. In another embodiment, the treatment catheter 102 may be configured for use in a valvuloplasty procedure or a catheter configured to deliver a device for repairing a native valve such as but not limited to an annuloplasty ring or band. Depending on the type of treatment or diagnostic catheter, the distal end portion thereof may include various types of medical components attached to or formed thereon. For example, if the catheter is a balloon catheter as described herein, the medical component at a distal end portion thereof is a balloon. In another embodiment, the catheter may be a diagnostic catheter and the medical component at a distal portion thereof includes a sensor or imaging device. In another embodiment, the catheter may be a treatment catheter such as a drug delivery catheter and the medical component at a distal portion thereof includes a drug delivery port.

As previously stated above, the scalloped configuration of the outer surface 162 of the locking interface is exemplary and the number of the plurality of grooves 164 and the plurality of ridges 166 may vary from that shown herein. For example, the locking interface 160 may include a fewer number of alternating ridges and grooves than shown, and the ridges and grooves are not required to be equally spaced around the outer surface 162 of the locking interface 160. The number of grooves 164 are not required to match or equal the number of ridges 166. In an embodiment, the locking interface may include single raised portion or ridge and at least two grooves formed on the outer surface. In another embodiment, the locking interface may include a single groove and at least two raised portions or ridges formed on the outer surface.

With respect to FIGS. 20-22, an alternative embodiment is shown in which the locking interface includes a single longitudinally extending ridge for interfacing with the collet 142. More particularly, a locking interface 2060 is shown that includes single ridge 2066 and a pair of adjacent grooves 2064 formed on an outer surface 2062 thereof. In this embodiment, the single ridge 2066 is formed between the pair of adjacent grooves 2064. Each groove 2064 of the pair of adjacent grooves 2064 is longitudinally extending, and extends at least the length of the collet 142 disposed thereover. Similarly, the single ridge 2066 is longitudinally extending, and extends at least the length of the collet 142 disposed thereover.

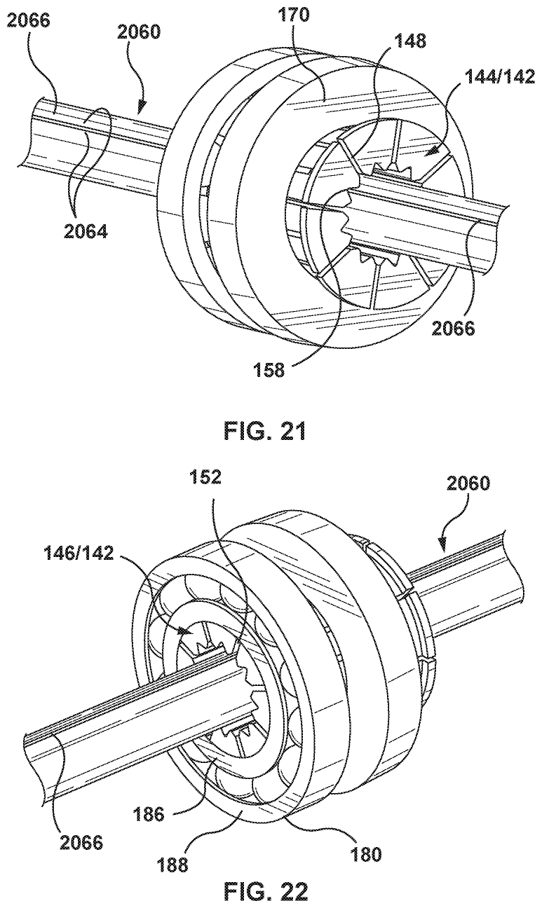

The collet 142 is disposed over the locking interface 2060. At the first end 144 of the collet 142, as best shown in FIG. 21, the single ridge 2066 of the locking interface 2060 aligns with a valley 158 of the inner surface of the collet 142. At the second end 146 of the collet 142, as best shown in FIG. 22, the single ridge 2066 of the locking interface 2060 aligns with a slot 152 of the second plurality of slots 152.

When the locking collar 170 is in the second or engaged position on the collet 142, the locking collar 170 radially compresses the collet 142 and thereby prevents or restricts rotation of the treatment catheter 102. With reference to FIG. 21, when the first end 144 of the collet 142 is radially compressed by the locking collar 170, the single ridge 2066 of the locking interface 2060 extends into or is received within a valley 158 of the inner surface of the collet 142. With the single ridge 2066 of the locking interface 2060 protruding into a valley 158 of the collet 142, the locking interface 160 and treatment catheter 102 attached thereto is prevented from rotating because the single ridge 2066 is disposed within the valley 158, between two adjacent peaks 156 of the collet 142. However, when the locking collar 170 is in the second or engaged position on the collet 142, the locking collar 170 still permits axial translation of the treatment catheter 102 because the single ridge 2066 of the locking interface 2060 is permitted to slide within the valley 158 of the collet 142.

When the locking bearing 180 is in the second or engaged position on the collet 142, the locking bearing 180 radially compresses the collet 142 and thereby prevents or restricts axial translation of the treatment catheter 102. With reference to FIG. 22, when the midportion of the collet 142 is radially compressed by the locking bearing 180, the single ridge 2066 is wedged into a slot 152 of the second plurality of slots 152. With the single ridge 2066 is wedged into a slot 152, the locking interface 2060 and treatment catheter 102 attached thereto cannot move axially, or are not permitted to move axially, under normal operating conditions. Thus, when the locking bearing 180 is in the second or engaged position on the collet 142, the treatment catheter 102 is no longer permitted to be axially translated or moved in a longitudinal direction relative to the guide catheter 102. However, when the locking bearing 180 is in the second or engaged position on the collet 142, the locking bearing 180 still permits rotation of the treatment catheter 102 because the inner bearing race 186 rotates relative to the outer bearing race 188. More particularly, with the single ridge 2066 is wedged into a slot 152, the collet 142 is mechanically interlocked with the locking interface 2060. When a user rotates the treatment catheter 102 (thus rotating the locking interface 2060 attached thereto), the assembly of the collet 142 and the inner bearing race 186 rotates therewith. Although the locking bearing 180 is radially compressing the midportion of the collet 142, the locking bearing 180 still permits rotation of the collet since the locking bearing 180 is a ball bearing. The inner bearing race 186 and balls 190 rotate with the collet 142, while outer bearing race 188 stays stationary.

The configuration of the inner surface of the collet 142 is also exemplary and the number of the peaks and valleys formed thereon may vary from that shown herein. For example, the collet 142 may include a fewer number of alternating peaks and valleys than shown, and the peaks and valleys are not required to be equally spaced around the inner surface of the collet 142. The number of peaks are not required to match or equal the number of grooves. In an embodiment, the collet may include a pair of peaks and a single groove formed on the inner surface thereof.

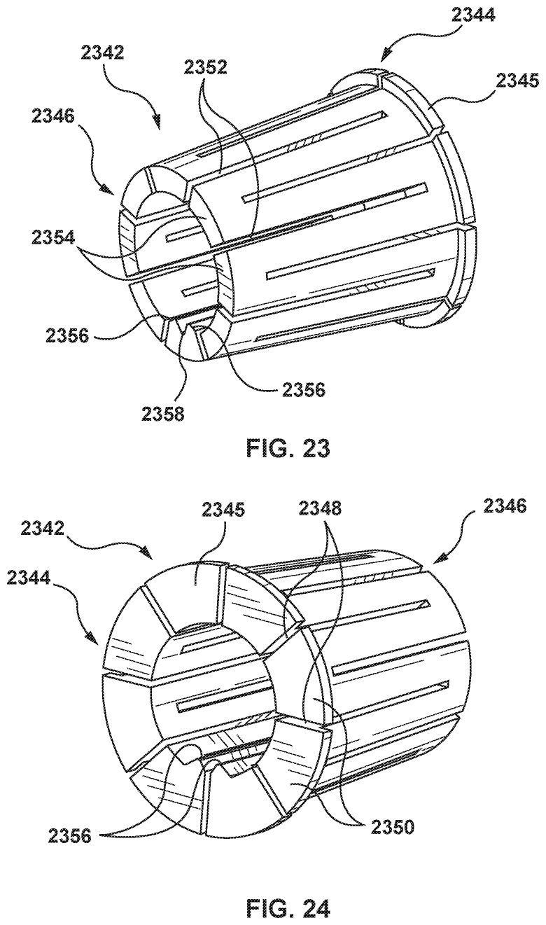

For example, FIGS. 23-26 illustrate an alternative embodiment of a collet 2342 for interfacing with the locking interface 160. More particularly, the collet 2342 is a generally tubular element having a first end 2344 and a second or opposing end 2346. FIG. 23 is a perspective end view from the first end 2344 of the collet 2342, and FIG. 24 is a perspective end view from the second end 2346 of the collet 2342. Similar to collet 142, a flange 2345 is formed on the first end 2344 of the collet 2342 and the wall thickness of the collet 2342 tapers from a first wall thickness at the first end 2344 to a second wall thickness at the second end 2346.

The collet 2342 includes a first plurality of slots 2348 extending from the first end 2344, towards the second end 2346. The first plurality of slots 2348 do not extend to the second end 2346, and therefore form a first plurality of integral tabs 2350 that circumferentially spaced apart around the first end 2344 of the collet 2342. Each tab 2350 of the first plurality of integral tabs 2350 is defined by a pair of adjacent slots 2348 of the first plurality of slots 2348. The first plurality of slots 2348 permit the first end 2344 of the collet 2342 to be radially compressible. When radially compressed, the width of the slots 2348 decreases until adjacent tabs 2350 of the first plurality of integral tabs 2350 are wedged against each other.

The collet 2342 similarly includes a second plurality of slots 2352 extending from the second end 2346, towards the first end 2344. The second plurality of slots 2352 do not extend to the first end 2344, and therefore form a second plurality of integral tabs 2354 that circumferentially spaced apart around the second end 2346 of the collet 2342. Each tab 2354 of the second plurality of integral tabs 2354 is defined by a pair of adjacent slots 2352 of the second plurality of slots 2352. The second plurality of slots 2352 permit the second end 2346 of the collet 2342 to be radially compressible. When radially compressed, the width of the slots 2352 decreases until adjacent tabs 2354 of the second plurality of integral tabs 2354 are wedged against each other. The first plurality of slots 2348 are circumferentially offset from the second plurality of slots 2352, with each slot 2348 extending approximately half-way between a pair of adjacent slots 2352 and each slot 2352 extending approximately half-way between a pair of adjacent slots 2348.

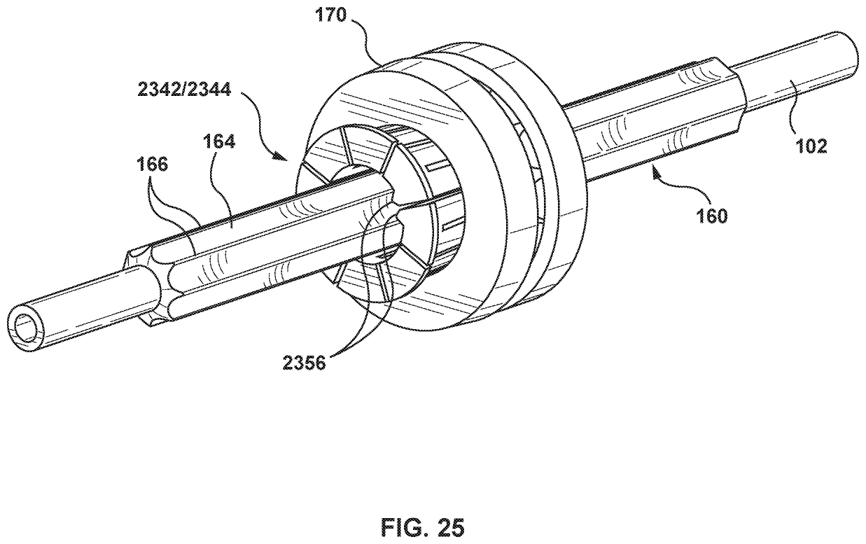

The collet 2342 includes an inner surface with a pair of peaks 2356 and a valley 2358. The valley 2358 is formed between the adjacent peaks 2356. In this embodiment, two adjacent tabs 2350 of the plurality of tabs 2350 each include a single peak 2356 of the pair of peaks 2356 and a single tab 2354 of the second plurality of integral tabs 2354 includes the pair of peaks 2356 and the valley 2358 formed therebetween. As best shown on FIG. 25, which is a perspective end view of the first end 2344 of the collet 2342 and the locking interface 160 disposed therein, at the first end 2344 of the collet 2342, the pair of peaks 2356 is aligned within a groove 164 of the locking interface 160 at the first end 2344 of the collet 2342. When the locking collar 170 is in the second or engaged position on the collet 2342, the locking collar 170 radially compresses the collet 2342 and thereby prevents or restricts rotation of the treatment catheter 102. When the first end 2344 of the collet 2342 is radially compressed by the locking collar 170, the pair of peaks 2356 are compressed into the groove 164 such that the pair of peaks 2356 are sandwiched or disposed between two ridges 166 of the locking interface 160. With the pair of peaks 2356 compressed into the groove 164, the locking interface 160 and treatment catheter 102 attached thereto is prevented from rotating. Thus, when the locking collar 170 is in the second or engaged position on the collet 2342, the treatment catheter 102 is no longer permitted to be rotated relative to the guide catheter 102. However, when the locking collar 170 is in the second or engaged position on the collet 2342, the locking collar 170 still permits axial translation of the treatment catheter 102 because the pair of peaks 2356 compressed into the groove 164 are permitted to slide within the groove 164.

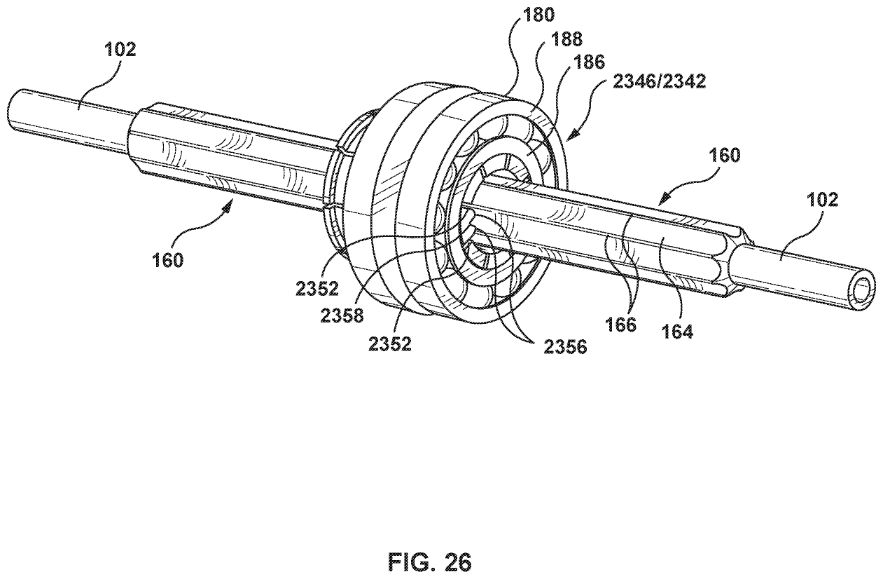

As best shown on FIG. 26, which is a perspective end view of the second end 2346 of the collet 2342 and the locking interface 160 disposed therein, at the second end 2346 of the collet 2342, the single tab 2354 of the second plurality of integral tabs 2354 which includes the pair of peaks 2356 and the valley 2358 formed therebetween is aligned with a groove 164 of the locking interface. When the locking bearing 180 is in the second or engaged position on the collet 2342, the locking bearing 180 radially compresses the collet 2342 and thereby prevents or restricts axial translation of the treatment catheter 102. When the midportion of the collet 2342 is radially compressed by the locking bearing 180, the single tab of the second plurality of integral tabs 2354 that includes the pair of peaks 2356 and the valley 2358 formed therebetween is wedged into a groove 164 of the locking interface 160. With slots 2352 on either side of this single tab 2354, the single tab of the second plurality of integral tabs 2354 which includes the pair of peaks 2356 and the valley 2358 formed therebetween is pushed into a groove 164 of the locking interface 160 and the midportion of the collet 2342 is radially compressed by the locking bearing 180. With the single tab of the second plurality of integral tabs 2354 which includes the pair of peaks 2356 and the valley 2358 formed therebetween wedged into a groove 164, the locking interface 160 and treatment catheter 102 attached thereto cannot move axially, or are not permitted to move axially, under normal operating conditions. Thus, when the locking bearing 180 is in the second or engaged position on the collet 2342, the treatment catheter 102 is no longer permitted to be axially translated or moved in a longitudinal direction relative to the guide catheter 102.

However, when the locking bearing 180 is in the second or engaged position on the collet 2342, the locking bearing 180 still permits rotation of the treatment catheter 102 because the inner bearing race 186 rotates relative to the outer bearing race 188. More particularly, with the single tab of the second plurality of integral tabs 2354 which includes the pair of peaks 2356 and the valley 2358 formed therebetween wedged into a groove 164 of the locking interface 160, the collet 2342 is mechanically interlocked with the locking interface 160. When a user rotates the treatment catheter 102 (thus rotating the locking interface 160 attached thereto), the assembly of the collet 2342 and the inner bearing race 186 rotates therewith. Although the locking bearing 180 is radially compressing the midportion of the collet 2342, the locking bearing 180 still permits rotation of the collet since the locking bearing 180 is a ball bearing. The inner bearing race 186 and balls 190 rotate with the collet 2342, while outer bearing race 188 stays stationary.

While various embodiments according to the present invention have been described above, it should be understood that they have been presented by way of illustration and example only, and not limitation. It will be apparent to persons skilled in the relevant art that various changes in form and detail can be made therein without departing from the spirit and scope of the invention. Thus, the breadth and scope of the present invention should not be limited by any of the above-described exemplary embodiments, but should be defined only in accordance with the appended claims and their equivalents. It will also be understood that each feature of each embodiment discussed herein, and of each reference cited herein, can be used in combination with the features of any other embodiment. All patents and publications discussed herein are incorporated by reference herein in their entirety.

1. A system comprising:
a handle;
a sheath attached to and distally extending from the handle; and
a tubular component slidingly disposed within the sheath,
wherein the handle includes a locking mechanism having an unlocked state in which the tubular component is permitted to slide freely in a longitudinal direction relative to the sheath and is permitted to rotate freely in a circumferential direction relative to the sheath, a locked state in which the tubular component is not permitted to slide freely in the longitudinal direction relative to the sheath and is not permitted to rotate freely in the circumferential direction relative to the sheath, a first semi-locked state in which the tubular component is permitted to slide freely in a longitudinal direction relative to the sheath and is not permitted to rotate freely in a circumferential direction relative to the sheath, and a second semi-locked state in which the tubular component is not permitted to slide freely in a longitudinal direction relative to the sheath and is permitted to rotate freely in a circumferential direction relative to the sheath.

2. The system of claim 1, wherein the locking mechanism includes a collet, a locking collar, and a locking bearing, each of the locking collar and the locking bearing being slidingly disposed over the collet.

3. The system of claim 2, further comprising a locking interface on an outer surface of the tubular component, the locking interface having an outer surface including a plurality of grooves and a plurality of ridges, each groove formed between a pair of adjacent ridges, wherein the collet is disposed over the locking interface and the collet has an inner surface with a sinusoidal configuration including a plurality of peaks and a plurality of valleys, each valley formed between a pair of adjacent peaks, wherein each groove of the locking interface is configured to receive a pair of adjacent peaks of the collet.

4. The system of claim 2, wherein each of the locking collar and the locking bearing is configured to be axially translated by a user between at least a non-engaged position on the collet and an engaged position on the collet.

5. The system of claim 4, wherein each of the locking collar and the locking bearing is disposed in the non-engaged position on the collet when the locking mechanism is in the unlocked state.

6. The system of claim 4, wherein each of the locking collar and the locking bearing is disposed in the engaged position on the collet when the locking mechanism is in the locked state.

7. The system of claim 4, wherein the locking collar is disposed in the engaged position and the locking bearing is disposed in the non-engaged position on the collet when the locking mechanism is in the first semi-locked state.

8. The system of claim 4, wherein the locking collar is disposed in the non-engaged position on the collet and the locking bearing is disposed in the engaged position on the collet when the locking mechanism is in the second semi-locked state.

9. The system of claim 1, where the handle further includes an indexing component configured to output a tactile click at one or more defined degrees of rotation of the tubular component.

10. A system comprising:
a treatment catheter; and
a guide catheter including a sheath configured to slidingly receive the treatment catheter and a handle coupled to the sheath, the handle including a locking mechanism including an unlocked state in which the treatment catheter is permitted to slide freely in a longitudinal direction relative to the sheath and is permitted to rotate freely in a circumferential direction relative to the sheath, a locked state in which the treatment catheter is not permitted to slide freely in the longitudinal direction relative to the sheath and is not permitted to rotate freely in the circumferential direction relative to the sheath, a first semi-locked state in which the treatment catheter is permitted to slide freely in a longitudinal direction relative to the sheath and is not permitted to rotate freely in a circumferential direction relative to the sheath, and a second semi-locked state in which the treatment catheter is not permitted to slide freely in a longitudinal direction relative to the sheath and is permitted to rotate freely in a circumferential direction relative to the sheath.

11. The system of claim 10, wherein an outer surface of the treatment catheter includes a locking interface thereon, the locking interface having an outer surface including a plurality of grooves and a plurality of ridges, each groove formed between a pair of adjacent ridges, and wherein the locking mechanism is disposed over the locking interface and has an inner surface with a sinusoidal configuration including a plurality of peaks and a plurality of valleys, each valley formed between a pair of adjacent peaks, wherein each groove of the locking interface is configured to receive a pair of adjacent peaks of the locking mechanism.

12. The system of claim 11, wherein the locking mechanism includes a collet, a locking collar, and a locking bearing, each of the locking collar and the locking bearing being slidingly disposed over the collet.

13. The system of claim 12, wherein the collet includes the inner surface including the plurality of peaks and the plurality of valleys.

14. The system of claim 12, wherein each of the locking collar and the locking bearing is configured to be axially translated by a user between at least a non-engaged position on the collet and an engaged position on the collet.

15. The system of claim 14, wherein each of the locking collar and the locking bearing is disposed in the non-engaged position on the collet when the locking mechanism is in the unlocked state.

16. The system of claim 15, wherein each of the locking collar and the locking bearing is disposed in the engaged position on the collet when the locking mechanism is in the locked state.

17. The system of claim 10, where the handle further includes an indexing component configured to output a tactile click at one or more defined degrees of rotation of the treatment catheter.

18. A system comprising:
a catheter, wherein an outer surface of the catheter including a locking interface having an outer surface including a plurality of grooves and a plurality of ridges, each groove formed between a pair of adjacent ridges; and
a locking mechanism disposed over the locking interface, wherein the locking mechanism includes a collet, a locking collar, and a locking bearing, each of the locking collar and the locking bearing being slidingly disposed over the collet, and
wherein the collet has an inner surface with a sinusoidal configuration including a plurality of peaks and a plurality of valleys, each valley formed between a pair of adjacent peaks, and
wherein each groove of the locking interface is configured to receive a pair of adjacent peaks of the collet, and
wherein at least one of the locking collar and the locking bearing is configured to be axially translated by a user between at least a non-engaged position on the collet and an engaged position on the collet.

19. (canceled)

20. (canceled)

21. The system of claim 18, each of the locking collar and the locking bearing is configured to be axially translated by the user between at least a non-engaged position on the collet and an engaged position on the collet.

22. The system of claim 18, wherein only the locking collar is configured to be axially translated by the user between at least a non-engaged position on the collet and an engaged position on the collet.
